(19) United States
(12) Patent Application Publication
(10) Pub. No.: US 2022/0204620 A1
(43) Pub. Date: Jun. 30, 2022

(54) MEANS AND METHODS FOR TREATING BURKITT LYMPHOMA OR LEUKEMIA

(71) Applicant: AMGEN RESEARCH (MUNICH) GMBH, Munich (DE)

(72) Inventors: Gerhard Zugmaier, Munich (DE); Max Topp, Würzburg (DE)

(21) Appl. No.: 17/605,061

(22) PCT Filed: Apr. 29, 2020
(86) PCT No.: PCT/EP2020/061851

§ 371 Date: Oct. 20, 2021

Related U.S. Application Data
(60) Provisional application No. 62/840,562, filed on Apr. 30, 2019.

(51) Int. Cl. C07K 16/28; A61P 35/00

(52) U.S. Cl. CPC C07K 16/2809; C07K 16/2803; C07K 2317/622; C07K 2317/31; A61P 35/00

(57) ABSTRACT

The present invention relates to a method of treating Burkitt lymphoma or leukemia in a patient, the method comprising administering to said patient a composition comprising a CD19×CD3 bispecific antibody in at least one treatment cycle, wherein the treatment cycle comprises administering a first dose of 15 μg/m²/d of the CD19×CD3 bispecific antibody for a first period of time of 4 days, and administering after the first dose a second dose of 60 Kg/m²/d of the CD19×CD3 bispecific antibody for a second period of time, wherein the second period of time exceeds the first period of time.

### FIELD OF THE INVENTION

The present invention relates to a method of treating Burkitt lymphoma or leukemia in a patient, the method comprising administering to said patient a composition comprising a CD19×CD3 bispecific antibody in at least one treatment cycle, wherein the treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time.

### BACKGROUND OF THE INVENTION

Burkitt lymphoma (BL) or leukemia are highly aggressive B-cell malignancies characterized clinically by a rapid growth phase often with extranodal presentation including frequent central nervous system (CNS) involvement. Burkitt lymphoma as aggressive neoplasm is classified as a Non-Hodgkin's Lymphoma (NHL) often presenting in extranodal sites or as an acute leukemia (deLeval et al. 2009, Hematology/Oncology Clinics of North America; Blum et al. 2004, Blood Journals). Originally thought to represent 2 different lymphoproliferative disorders, BL was historically classified as a small noncleaved cell lymphoma in patients with a solid tumor or nodal mass and as L3 acute lymphoblastic leukemia (FAB [French-American-British] L3 ALL) in patients with greater than 25% bone marrow involvement. However, on the basis of shared molecular and genetic features, the World Health Organization (WHO) Classification of Lymphoid Diseases recognizes the lymphoma and leukemic phases of BL as a single entity; a mature B-cell neoplasm, subtype Burkitt lymphoma/Burkitt cell leukemia. Even though both diseases include one disease entity, the response criteria need assessment of the clinical entities of lymphoma and leukemia.

Burkitt lymphoma is subdivided into three different categories based on epidemiological observations: endemic BL (eBL), sporadic BL (sBL), and HIV-associated BL. Its incidence is particularly high in equatorial Africa and Northeast Brazil (Magrath J Pediatr Hematol Oncol Am 1991; 13:. 222-46; Sandlund et al Leukemia 1997; 11: 743) and accounts for 40-50% of lymphomas non-Hodgkin in most industrialized countries (Murphy et al J Clin Oncol 1989; 7:18693; Wilson et al Cancer 1984; 53:. 1695-704).

The common characteristic of virtually all BL is translocation of the MYC proto-oncogene to an immunoglobulin (Ig) locus (Brady et al. 2008, Postgraduate Medical Journal). MYC encodes the c-myc transcription factor which was first discovered nearly thirty years ago as a cellular homologue of an avian retroviral oncogene (Vennstrom et al. 1982, Journal of Virology).

BL is a tumor fastest growing (Bouffet et al Eur J Cancer 1991; 27: 504-9) so that the tumor doubles in size every 24 hours (Kearns et al Int J Pediatr Otorhinolaryngol 1986, 12: 73-84; Ziegler N Engl J Med 1977; 297: 75-80). Because of these characteristics, this lymphoma is still a very rapid clinical course without treatment and death occurs within months. BL requires, therefore, a very rapid diagnosis before starting a specific treatment.

Therapy BL is based on a very aggressive and intensive chemotherapy including various combinations of cyclophosphamide, vincristine, doxorubicin, methotrexate, ifosfamide, etoposide, and cytarabine and intrathecal chemotherapy or systemic administration of chemotherapeutic agents capable of cross the blood (Divine et al Ann Oncol 2005; 16:. 1928-1935; Magrath et al J Clin Oncol 1996; 14. 92534; Mead et al Ann Oncol 2002; 13:. 1264-1274; Pees et al. Ann Hematol 1992; 65: 201-5; Thomas et al J Clin Oncol 1999; 17:. 2461-70) due to the high risk of central nervous system (Bishop et al Cancer Invest 2000; 18:. 574-83). Unfortunately, the toxicity of this aggressive therapy is huge because of the side effects involved, including neurotoxicity, hematologic toxicity, severe mucositis, heart disease and infertility (Patte et al Blood 2007; 109:. 2773-80).

The standard treatment was and still is chemotherapy based on CHOP. CHOP consists of four chemotherapy drugs—Cyclophosphamide (also called Cytoxan/Neosar), Doxorubicin (also called Hydroxydaunorubicin) (or Adriamycin), Vincristine (Oncovin) and Prednisolone. However, though the CHOP treatment was and is usually applied, the development of new treatment regimens including M-BACOD (methotrexate, bleomycin, doxorubicin, cyclophosphamide, vincristine, and dexamethasone), MACOP-B (methotrexate with leucovorin rescue doxorubicin, cyclophosphamide, vincristine, prednisone, and bleomycin) and ProMACE/CytaBOM (cyclophosphamide, doxorubicin, etoposide cytozar, bleomycin, vincristine, methotrexate prednisone) were reported to achieve results that seemed much better than had been observed with CHOP. These so-called third-generation regimens appeared to represent an important advance in therapy until an intergroup trial carried out in the United States demonstrated no superiority over CHOP.

Nevertheless, the CHOP therapy was expanded to a combination of chemotherapy and immunotherapy, i.e. R-CHOP. R-CHOP is a combination of drugs used in chemotherapy for aggressive Non-Hodgkin Lymphomas (NHL). It adds the drug Rituximab—a monoclonal antibody against CD20, to the standard combination called CHOP.

A commonly applied R-CHOP treatment regime is as follows: Rituximab is administered as an infusion over a few hours on the first day of treatment, while the drugs of the CHOP regimen may be started the next day. The entire course is usually repeated every three weeks for 6-8 cycles. The first three drugs of the CHOP chemotherapy regimen are usually given as injections or infusions in veins on a single day, while prednisolone is taken as pills for five days. Each cycle is repeated every 3 weeks for 6-8 cycles.

DHAP is a further combination of chemotherapeutic agents that is sometimes applied for the treatment of Burkitt lymphoma or leukemia. The DHAP chemotherapeutics include: Dexamethasone, cytarabine, which is sometimes called Ara-C and cisplatin, which contains platinum. DHAP can also be combined with Rituximab (tradename Rituxan®), i.e., R-DHAP.

ICE is another combination of chemotherapeutic agents that is sometimes applied for the treatment of Burkitt lymphoma or leukemia and named after the initials of the chemotherapy drugs used, which are: Ifosfamide, Carboplatin, and Etoposide. R-ICE also includes the monoclonal antibody drug Rituximab.

However, despite this major therapeutic advance, a significant proportion of patients will relapse or remain refractory to initial chemoimmunotherapy. Accordingly, there is a need for alternative and/or adjuvant therapies for the treatment of Burkitt lymphoma or leukemia, since until now there is no standard therapy established and almost every patient will die from progression of the disease. The technical problem underlying the present application is thus to comply with these needs. The technical problem is solved by providing the embodiments reflected in the claims, described in the description and illustrated in the examples and figures that follow.

### SUMMARY OF THE INVENTION

The present inventors have developed a novel treatment for Burkitt lymphoma or leukemia, preferably for patients suffering from relapsed and/or refractory Burkitt lymphoma or leukemia.

In particular, the present inventors have developed a specific treatment regimen comprising a CD19×CD3 bispecific antibody for effectively treating Burkitt lymphoma or leukemia, in particular relapsed and/or refractory Burkitt lymphoma or leukemia.

Because Burkitt lymphoma or leukemia has a higher proliferation rate than B-precursor acute lymphomblastic leukemia (BPC-ALL) and shows extramedullary involvement more commonly than BPC-ALL, it was specifically found that a first lower dose should be administered for a short treatment period of 4 days followed by a higher maintenance dose being applied to the patient in need thereof. This lower dose should be higher than the one adminstered in ALL. The target dose should be the one adminstered in patients with NHL. The single step adminstered in ALL should be kept. This treatment regimen addresses the characteristics of this indication, which are a mix of characteristics of NHL and ALL.

In sum, the present inventors were able to demonstrate in three patients with Burkitt lymphoma/leukemia (particularly with relapsed and/or refractory Burkitt lymphoma or leukemia) that this dosing schema according to the present invention is feasible and safe.

Accordingly, in a first aspect, the present invention relates to a method of treating Burkitt lymphoma or leukemia in a patient, the method comprising administering to said patient a composition comprising a CD19×CD3 bispecific antibody in at least one treatment cycle, wherein the treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time.

Preferably, according to the method of the present invention the second period of time exceeds 18 days. Even more preferably, according to the method of the present invention the second period of time is between 18 and 60 days, with 24 days being preferred.

The present invention may also comprise the method of the present invention, further comprising administering after the first treatment cycle at least a second treatment cycle.

Preferably, according to the method of the present invention the second treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time. More preferably, according to the method of the present invention, the second period of time exceeds 18 days. Even more preferably, according to the method of the present invention the second period of time is between 18 and 60 days, with 24 days being preferred.

Preferably, according to the method of the present invention the second treatment cycle is initiated at least 5 days after completing the first treatment cycle, preferably 10 days after completing the first treatment cycle.

The present invention may also envisage the method of the present invention, further comprising administering after the first and second treatment cycle at least a third treatment cycle, preferably five additional treatment cycles.

According to the method of the present invention, the route of administration of the first and the second dose of the CD19×CD3 bispecific antibody is preferably intravenous, even more preferably continuous intravenous.

Preferably, according to the method of the present invention, Burkitt lymphoma or leukemia is relapsed and/or refractory Burkitt lymphoma or leukemia. According to the method of the present invention, Burkitt lymphoma or leukemia is preferably a CD19 positive Burkitt lymphoma or leukemia.

Preferably, according to the method of the present invention the CD19×CD3 bispecific antibody is a bispecific single chain antibody. Even more preferably, said antibody is MT103/AMG103.

According to the method of the present invention, administering pentosane polysulfate (PPS) prior to the first dose and prior to the second dose of the CD19×CD3 bispecific antibody of each treatment cycle to said patient is preferably further comprised. Pentosane polysulfate is preferably administered between 24 and 2 hours prior to the first dose and prior to the second dose of the CD19×CD3 bispecific antibody, more preferably 24 hours prior to the first dose and prior to the second dose of the CD19×CD3 bispecific antibody. Preferably, pentosane polysulfate administration is then continued for at least 3 consecutive days after administration of the first dose and the second dose of the CD19×CD3 bispecific antibody. Accordingly, pentosane polysulfate administration is preferably continued for 7, 8, 9 or 10 consecutive days after administration of the first dose of the CD19×CD3 bispecific antibody, more preferably for 7 consecutive days after administration of the first dose of the CD19×CD3 bispecific antibody. Preferably, pentosane polysulfate is administered in a dose between 100 and 600 mg per day. More preferably, pentosane polysulfate is administered in a dose of 300 mg per day. In a most preferred embodiment of the present invention, pentosane polysulfate is administered in three doses of 100 mg per day. According to the method of the present invention, pentosane polysulfate is preferably administered orally to said patient.

Preferably, according to the method of the present invention the treatment based on the method of the present invention results in a MRD negative status of the Burkitt lymphoma or leukemia patient.

Preferably, according to the method of the present invention the patient is characterized by a B:T cell ratio of less than 1:8.

Preferably, according to the method of the present invention said patient is a mammal, preferably a primate, most preferably a human being.

Accordingly, in a second aspect, the present invention relates to a composition comprising a CD19×CD3 bispecific antibody for use in a method of treating Burkitt lymphoma or leukemia in a patient, wherein the method comprises administering to said patient the composition in at least one treatment cycle, wherein the treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time.

Preferably, according to the composition of the present invention the second period of time exceeds 18 days. Even more preferably, according to the composition of the present invention the second period of time is between 18 and 60 days, with 24 days being preferred.

The present invention may also comprise the composition of the present invention, further comprising administering after the first treatment cycle at least a second treatment cycle.

Preferably, according to the composition of the present invention the second treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time. More preferably, according to the composition of the present invention, the second period of time exceeds 18 days. Even more preferably, according to the composition of the present invention the second period of time is between 18 and 60 days, with 24 days being preferred.

Preferably, according to the composition of the present invention the second treatment cycle is initiated at least 5 days after completing the first treatment cycle, preferably 10 days after completing the first treatment cycle.

The present invention may also envisage the composition of the present invention, further comprising administering after the first and second treatment cycle at least a third treatment cycle, preferably five additional treatment cycles.

According to the composition of the present invention, the route of administration of the first and the second dose of the CD19×CD3 bispecific antibody is preferably intravenous, even more preferably continuous intravenous.

Preferably, according to the composition of the present invention, Burkitt lymphoma or leukemia is relapsed and/or refractory Burkitt lymphoma or leukemia. According to the composition of the present invention, Burkitt lymphoma or leukemia is preferably a CD19 positive Burkitt lymphoma or leukemia.

Preferably, according to the composition of the present invention the CD19×CD3 bispecific antibody is a bispecific single chain antibody. Even more preferably, said antibody is MT103/AMG103.

According to the composition of the present invention, administering pentosane polysulfate prior to the first dose and prior to the second dose of each treatment cycle to said patient is preferably further comprised. Pentosane polysulfate is preferably administered between 24 and 2 hours prior to the first dose and prior to the second dose of the CD19×CD3 bispecific antibody, more preferably 24 hours prior to the first dose and prior to the second dose of the CD19×CD3 bispecific antibody. Preferably, pentosane polysulfate administration is then continued for at least 3 consecutive days after administration of the first dose and the second dose of the CD19×CD3 bispecific antibody. Accordingly, pentosane polysulfate administration is preferably continued for 7, 8, 9 or 10 consecutive days after administration of the first dose of the CD19×CD3 bispecific antibody, more preferably for 7 consecutive days after administration of the first dose of the CD19×CD3 bispecific antibody. Preferably, pentosane polysulfate is administered in a dose between 100 and 600 mg per day. More preferably, pentosane polysulfate is administered in a dose of 300 mg per day. In a most preferred embodiment of the present invention, pentosane polysulfate is administered in three doses of 100 mg per day. According to the composition of the present invention, pentosane polysulfate is preferably administered orally to said patient.

Preferably, according to the composition of the present invention the treatment based on the composition of the present invention results in a MRD negative status of the Burkitt lymphoma or leukemia patient.

Preferably, according to the composition of the present invention the patient is characterized by a B:T cell ratio of less than 1:8.

Preferably, according to the composition of the present invention said patient is a mammal, preferably a primate, most preferably a human being.

Accordingly, in a third aspect, the present invention relates to the use of a composition comprising a CD19×CD3 bispecific antibody for the manufacture of a medicament for the treatment of Burkitt lymphoma or leukemia in a patient, the treatment comprising administering to said patient the composition in at least one treatment cycle, wherein the treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time.

### DETAILED DESCRIPTION OF THE INVENTION

In order to overcome some of the shortcomings of the means described so far in the prior art for the treatment of Burkitt lymphoma or leukemia, the inventors provide herein a promising new treatment regimen for treating Burkitt lymphoma or leukemia.

Burkitt lymphoma or leukemia are highly aggressive B-cell malignancies characterized clinically by a rapid growth phase often with extranodal presentation including frequent central nervous system (CNS) involvement.

Burkitt lymphoma (BL) as aggressive neoplasm is classified as a Non-Hodgkin's Lymphoma (NHL) often presenting in extranodal sites or as an acute leukemia (deLeval et al. 2009, Hematology/Oncology Clinics of North America; Blum et al. 2004, Blood Journals). Originally thought to represent 2 different lymphoproliferative disorders, BL was historically classified as a small noncleaved cell lymphoma in patients with a solid tumor or nodal mass and as L3 acute lymphoblastic leukemia (FAB [French-American-British] L3 ALL) in patients with greater than 25% bone marrow involvement. However, on the basis of shared molecular and genetic features, the World Health Organization (WHO) Classification of Lymphoid Diseases recognizes the lymphoma and leukemic phases of BL as a single entity; a mature B-cell neoplasm, subtype Burkitt lymphoma/Burkitt cell leukemia. Burkitt lymphoma retains features of both the leukemia's and lymphomas but are derived from immature progenitor cells and require therapy similar to that used for acute lymphoblastic leukemia ALL. Even though both diseases include one disease entity according to WHO classification, the response criteria need assessment of the clinical entities of lymphoma and leukemia as will be described elsewhere herein.

The abnormally rapid proliferation of B-lymphocytes, which is characteristic of BL, has been attributed to certain changes in gene expression. The oncogene in question is the Myc gene situated on chromosome 8. Eighty percent of BL cases harbor t(8;14), resulting in the juxtaposition of the c-myc gene on chromosome 8 with IgH enhancer elements on chromosome 14. Translocations occurring between chromosomes 2 and 8, t(2;8)(p12;q24), or chromosomes 8 and 22, t(8;22)(q24;q11), place the c-myc gene adjacent to either K or A light chain loci and enhancer elements.

The precise symptoms may vary according to the different types of Burkitt lymphoma and may include, but are not limited to, the following indications such as loosening of the molars or premolars in children, sweating profusely at night, fever or flu, loss of appetite and loss of weight, diarrhea and/or bowel obstruction and/or bleeding, anemia and impairment in blood clotting, protrusion of the eyeballs, enlargement of the thyroid gland, nausea and vomiting, or gastrointestinal bleeding.

In sum, the present invention opens a new avenue by developing a more effective therapy for Burkitt lymphoma or leukemia. In particular, relapsed and/or refractory patients, preferably pretreated with a CD20 directed antibody therapy as part of their previous chemotherapy regimentsm, can be treated with the method of the present invention.

Thus, the present invention provides in a first aspect a method of treating Burkitt lymphoma or leukemia in a patient, the method comprising administering to said patient a composition comprising a CD19×CD3 bispecific antibody in at least one treatment cycle, wherein the treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time.

Typically, the diagnosis of lymphoma, is generally done in a sample obtained from a patient suspected to develop and/or have a lymphoma, in particular Burkitt lymphoma.

In this regard, the term “sample” is intended any biological sample obtained from a human patient containing polynucleotides or polypeptides or portions thereof. Biological samples include body fluids (such as blood, serum, plasma, urine, saliva, synovial fluid and spinal fluid) and tissue sources found to malignant CD19 positive lymphocytes. Methods for obtaining tissue biopsies and body fluids from patients are well known in the art. Generally, a biological sample which includes peripheral blood mononuclear cells (PBMCs), in particular B cells and T cells is preferred as a source.

A sample which includes peripheral blood mononuclear cells (PBMCs), in particular B cells and T cells, is preferably taken from peripheral blood of a human patient. Other preferred samples are whole blood, serum, plasma or synovial fluid, with plasma or serum being most preferred.

Another preferred sample obtained from a patient is a lymph node biopsy. A lymph node biopsy is, for example, obtained with an excisional biopsy of an abnormal lymph node or a generous incisional biopsy of an involved organ. In some cases, cutting-needle biopsies can provide adequate tissue for diagnosis. In addition, an adequate bone marrow biopsy may be performed. Diagnosis can be supplemented by gene-expression profiling. More preferably, the diagnosis is preferably made by a hematopathologist with experience in diagnosing lymphomas, in particular Burkitt lymphoma or leukemia by, preferably applying the WHO classification of lymphoid neoplasma (see Table 1 on page 30 of the publication of Armitage in Blood (2007), Vol. 110 (1):29-36). It is sometimes also preferred to perform immunohistochemistry and on occasion to apply cytogenetics or fluorescent in situ hybridisation (FISH) in order to clarify an initial diagnosis.

Once the diagnosis is made and, preferably confirmed, additional tests such as restaging by re-biopsy by a further experienced hematopathologist and/or further imaging studies including computer tomography, ultra sound imaging, and/or PET scan of the chest, abdomen and/or pelvis, are performed to obtain more information about the extent to which the disease has spread in the body. This process is called staging. The results of these tests will help determine the most effective course of treatment.

A number of staging tests are available to help determine which areas of the body have been affected by follicular lymphoma. Tests that may be done include: CT scan, blood tests, bone marrow biopsy and/or PET scan.

Staging involves dividing patients into groups (stages) based upon how much of the lymphatic system is involved at the time of diagnosis. Staging helps determine a person's prognosis and treatment options.

Stages of lymphoma can be defined as follows:

Stage I—Only one lymph node region is involved, or only one lymph structure is involved.
Stage II—Two or more lymph node regions or lymph node structures on the same side of the diaphragm are involved.
Stage III—Lymph node regions or structures on both sides of the diaphragm are involved.
Stage IV—There is widespread involvement of a number of organs or tissues other than lymph node regions or structures, such as the liver, lung, or bone marrow.

When a stage is assigned, it also includes a letter, A or B, to denote whether fever, weight loss, or night sweats are present. “A” means these symptoms are not present; “B” means they are. For example, a person with stage 1B disease has evidence of cancer in one lymph node region and has “B” symptoms (fever, weight loss, or night sweats).

“Leukemias” are clonal neoplastic proliferations of immature hematopoietic cells that are characterized by aberrant or arrested differentiation. Leukemia cells accumulate in the bone marrow, ultimately replacing most of normal hematopoietic cells. This results in bone marrow failure and its consequences of anemia, hemorrhage and infection. Leukemia cells circulate into the blood and other tissues throughout the body. According to the present invention, Burkitt leukemia is a rare, fast-growing type of leukemia (blood cancer) in which too many white blood cells called B lymphocytes form in the blood and bone marrow. It may start in the lymph nodes as Burkitt lymphoma and then spread to the blood and bone marrow, or it may start in the blood and bone marrow without involvement of the lymph nodes.

In a preferred embodiment according to the present invention, Burkitt lymphoma or leukemia being treated by the methods and means of the present invention is relapsed and/or refractory Burkitt lymphoma or leukemia.

The term “relapsed Burkitt lymphoma or leukemia” as used herein denotes the return of signs and symptoms of Burkitt lymphoma or leukemia after a patient has enjoyed a remission. For example, after conventional Burkitt lymphoma or leukemia treatment using standard chemotherapy as known to the person skilled in the art and/or HSCT, preferably the patient receiving a CD20 directed antibody therapy as part of their chemotherapy regiments, a Burkitt lymphoma or leukemia patient may go into remission with no sign or symptom of Burkitt lymphoma or leukemia, remains in remission, but then suffers a relapse and has to be treated once again for Burkitt lymphoma or leukemia. Preferably, according to the present invention the term “relapsed” means that the duration of first remission is greater than 3 months.

The term “refractory Burkitt lymphoma or leukemia” as used herein means resistance of Burkitt lymphoma or leukemia to conventional or standard Burkitt lymphoma or leukemia therapy, such as chemotherapy and/or HSCT. The conventional or standard Burkitt lymphoma or leukemia therapy is not able to ultimately cure all patients. Preferably, according to the present invention the term “refractory” is defined by relapse within 3 months after prior remission.

Burkitt lymphoma or leukemia when referred to herein by the methods and means of the present invention comprises preferably malignant CD19 positive lymphocytes. Thus, in a preferred embodiment, the Burkitt lymphoma or leukemia according to the present invention is a CD19 positive Burkitt lymphoma or leukemia. “Malignant” describes lymphocytes (in particular B cells) that contribute to a progressively worsening disease, in particular Burkitt lymphoma or leukemia as described herein. Malignant CD19 positive lymphocytes (in particular B cells) are not self-limited in their growth, are capable of invading into adjacent tissues, and may be capable of spreading to distant tissues (metastasizing). Malignant when used herein is synonymous with cancerous. However, as “normal” (non-malignant) lymphocytes (in particular B cells) also express CD19, it is to be expected that the CD19×CD3 bispecific antibody also binds these normal lymphocytes (in particular B cells) and upon recruiting cytotoxic T cells (because of the second specificity of the bispecific CD19×CD13 antibody) depletes these normal B cells. Yet, it is expected that the population of these normal B cells is reconstituted in the absence of the CD19×CD3 bispecific antibody. It was observed by Leandro and co-workers that after their depletion by an anti-CD20 antibody, B cells were reconstituted in rheumatoid arthritis patients (Arthritis Rheum. 2006 February; 54(2):613-20). As CD20, likewise CD19 is expressed on almost all B cells, it can be expected that B cells upon depletion by the bispecific CD19×CD3 antibody are reconstituted, too.

“Treatment” or “treating” as used herein is defined as the application or administration of a CD19×CD3 bispecific antibody to a patient, or application or administration of CD19×CD3 bispecific antibody to an isolated tissue or cell line from a patient, where the patient has Burkitt lymphoma or leukemia or is at a risk of developing Burkitt lymphoma or leukemia, a symptom of Burkitt lymphoma or leukemia, or a predisposition towards Burkitt lymphoma or leukemia, where the purpose is to cure, heal, alleviate, relieve, alter, remedy, ameliorate, improve, or affect Burkitt lymphoma or leukemia, the symptoms of Burkitt lymphoma or leukemia, or the predisposition towards Burkitt lymphoma or leukemia. By “treatment” is also intended the application or administration of a pharmaceutical composition comprising the CD19×CD3 bispecific antibody to a patient, or application or administration of a pharmaceutical composition comprising the CD19×CD3 bispecific antibody, to an isolated tissue or cell line from a patient, who has a disease, a symptom of a disease, or a predisposition toward a disease, where the purpose is to cure, heal, alleviate, relieve, alter, remedy, ameliorate, improve, or affect the disease, the symptoms of the disease, or the predisposition toward the disease. The (pharmaceutical) composition applied in the present invention preferably comprises a “therapeutically effective amount” of a CD19×CD3 bispecific antibody.

The exact therapeutically effective dose of the CD19×CD3 bispecific antibody to treat Burkitt lymphoma or leukemia will be ascertainable by one skilled in the art using known techniques. As is known in the art and described elsewhere herein, adjustments for age, body weight, general health, sex, diet, drug interaction and the severity of the condition may be necessary, and will be ascertainable with routine experimentation by those skilled in the art. The therapeutic effect of the respective methods or method steps of the present invention is additionally detectable by all established methods and approaches which will indicate a therapeutic effect. It is, for example, envisaged that the therapeutic effect is detected by way of surgical resection or biopsy of an affected tissue/organ which is subsequently analyzed by way of immunohistochemical (IHC) or comparable immunological techniques. Alternatively it is also envisaged that the tumor markers in the serum of the patient (if present) are detected in order to diagnose whether the therapeutic approach is already effective or not. Additionally or alternatively it is also possible to evaluate the general appearance of the respective patient (fitness, well-being, decrease of tumor-mediated ailment etc.) which will also aid the skilled practitioner to evaluate whether a therapeutic effect is already there. The skilled person is aware of numerous other ways which will enable him or her to observe a therapeutic effect of the compounds of the present invention.

By “therapeutically effective dose”, “therapeutically effective amount” or “effective amount” of a composition comprising a CD19×CD3 bispecific antibody with respect to Burkitt lymphoma or leukemia is intended an amount of a CD19×CD3 bispecific antibody that, when administered brings about a positive therapeutic response with respect to treatment of a patient with Burkitt lymphoma or leukemia. Suitable dosages are described in more detail elsewhere herein. It also refers to, in one embodiment, an amount of the composition that delays, reduces, palliates, ameliorates, stabilizes, prevents and/or reverses one or more symptoms (e.g., clinical symptom, biochemical symptom, etc.) that are associated with Burkitt lymphoma or leukemia compared to in the absence of the composition. This includes using dosages and periods of time necessary, to achieve the desired therapeutic result. The term “delaying” symptoms refers to increasing the time period between exposure to the CD19×CD3 bispecific antibody and the onset of one or more symptoms as described herein. The term “eliminating” symptoms refers to 40, 50, 60, 70, 80, 90 or even 100% reduction of one or more symptoms as described herein. A therapeutically effective amount also includes one in which any toxic or detrimental effects of the composition are outweighed by therapeutically beneficial effects.

Thus, it is generally preferred that a composition comprising a CD19×CD3 bispecific antibody brings about a positive therapeutic response with respect to treatment of a patient with Burkitt lymphoma or leukemia.

By “positive therapeutic response” with respect to Burkitt lymphoma or leukemia is intended an improvement in Burkitt lymphoma or leukemia in association with the therapeutic activity of the CD19×CD3 bispecific antibody, and/or an improvement in the symptoms associated with Burkitt lymphoma or leukemia. That is, an anti-proliferative effect, the prevention of further tumor outgrowths, a reduction in tumor size, a reduction in the number of cancer cells, and/or a decrease in one or more symptoms associated with CD19-expressing cells can be observed. Thus, for example, a positive therapeutic response would refer to one or more of the following improvements in the disease: (1) a reduction in tumor size; (2) a reduction in the number of cancer (i.e., neoplastic) cells; (3) an increase in neoplastic cell death; (4) inhibition of neoplastic cell survival; (4) inhibition (i.e., slowing to some extent, preferably halting) of tumor growth; (5) inhibition (i.e., slowing to some extent, preferably halting) of cancer cell infiltration into peripheral organs; (6) inhibition (i.e., slowing to some extent, preferably halting) of tumor metastasis; (7) the prevention of further tumor outgrowths; (8) an increased patient survival rate; and (9) some extent of relief from one or more symptoms associated with Burkitt lymphoma or leukemia.

Positive therapeutic responses in any given malignancy can be determined by standardized response criteria specific to that malignancy. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation. In addition to these positive therapeutic responses, the patient undergoing therapy with the CD19×CD3 bispecific antibody may experience the beneficial effect of an improvement in the symptoms associated with the disease.

An improvement in the disease may be characterized as a complete response. By “complete response” is intended an absence of clinically detectable disease with normalization of any previously abnormal imaging studies such as radiographic studies. Such a response preferably persists for at least 4 to 8 weeks, sometimes 6 to 8 weeks or more than 8, 10, 12, 14, 16, 18 or 20 weeks or longer, following treatment according to the invention. Alternatively, an improvement in the disease may be categorized as being a partial response. By “partial response” is intended at least about a 50% decrease in all measurable tumor burden (i.e., the number of malignant cells present in the patient, or the measured bulk of tumor masses) in the absence of new lesions and persisting for 4 to 8 weeks or more than 8, 10, 12, 14, 16, 18 or 20 weeks or longer. A “complete response” does, however, not necessarily indicate that Burkitt lymphoma or leukemia has been cured, since a patient may relapse. However, if so, the patient can again be treated with a composition comprising a CD19×CD3 bispecific antibody as described herein. Detailed remission and response definitions for NHL patients are used according to Cheson et al., 1999, J. Clin. Oncol. April; 17(4):1244.

In some embodiments, the patient is pretreated, advantageously with chemotherapy such as CHOP or DHAP, experimental chemotherapy and/or chemoimmunotherapy such as R-CHOP, R-DHAP, R-ICE, R-VI PE, R-Treo/Flud or has undergone autologous stem cell therapy (SCT). Preferably, the patient is pretreated with a CD20 directed antibody therapy as part of their previous chemotherapy regiments.

By “pretreated” or “pretreatment” is intended the patient has received one or more other Burkitt lymphoma or leukemia therapies (i.e., been treated with at least one other Burkitt lymphoma or leukemia therapy) prior to receiving the composition comprising the CD19×CD3 bispecific antibody. “Pretreated” or “pretreatment” includes patients that have been treated with at least one other Burkitt lymphoma or leukemia therapy within 2 years, within 18 months, within 1 year, within 6 months, within 2 months, within 6 weeks, within 1 month, within 4 weeks, within 3 weeks, within 2 weeks, within 1 week, within 6 days, within 5 days, within 4 days, within 3 days, within 2 days, or even within 1 day prior to initiation of treatment with the composition comprising the CD19×CD3 bispecific antibody. It is not necessary that the patient was a responder to pretreatment with the prior Burkitt lymphoma or leukemia. Thus, the patient that receives the composition comprising the CD19×CD3 bispecific antibody could have responded, or could have failed to respond (i.e. Burkitt lymphoma or leukemia was refractory), to pretreatment with the prior Burkitt lymphoma or leukemia therapy, or to one or more of the prior Burkitt lymphoma or leukemia therapies where pretreatment comprised multiple Burkitt lymphoma or leukemia therapies.

Moreover, in other embodiments the patient is treated according to the means and methods of the present invention before s/he undergoes SCT.

In some embodiments and as described elsewhere herein, the patient is refractory to chemotherapy treatment or experimental chemotherapy treatment and/or in relapse after treatment with chemotherapy or experimental chemotherapy treatments.

In some of the foregoing embodiments, the patient is resistant to standard chemotherapeutic or experimental chemotherapy treatments.

When used herein a “composition comprising a CD19×CD3 bispecific antibody” encompasses preferably a pharmaceutical composition. Thus, according to the present invention the CD19×CD3 bispecific antibody is preferably in the form of a medicament. Accordingly, the term “pharmaceutical composition” and “medicament” when used herein are interchangeable.

In this specification the term “pharmaceutical” shall have its widest meaning and include compound(s) used in the treatment of Burkitt lymphoma or leukemia in a patient. Preferably, the compound used in the treatment of Burkitt lymphoma or leukemia is a CD19×CD3 bispecific antibody. Accordingly, a pharmaceutical composition preferably comprises a CD19×CD3 bispecific antibody and, optionally, a pharmaceutically acceptable carrier.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

Proper fluidity may be maintained, for example, by the use of coating materials, such as lecithin, by the maintenance of the required particle size in the case of dispersions, and by the use of surfactants.

Pharmaceutical compositions containing the antibodies may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. Pharmaceutical compositions of the present invention may also comprise isotonicity agents, such as sugars, polyalcohols such as mannitol, sorbitol, glycerol or sodium chloride in the compositions.

Pharmaceutically acceptable diluents include saline and aqueous buffer solutions. The pharmaceutical compositions containing the antibodies may also contain one or more adjuvants appropriate for the chosen route of administration, such as preservatives, wetting agents, emulsifying agents, dispersing agents, preservatives or buffers, which may enhance the shelf life or effectiveness of the pharmaceutical composition. Compounds of the present invention may for instance be admixed with lactose, sucrose, powders (e.g., starch powder), cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol. Other examples of adjuvants are QS21, GM-CSF, SRL-172, histamine dihydrochloride, thymocartin, Tio-TEPA, monophosphoryl-lipid A/microbacteria compositions, alum, incomplete Freund's adjuvant, montanide ISA, ribi adjuvant system, TiterMax adjuvant, syntex adjuvant formulations, immune-stimulating complexes (ISCOMs), gerbu adjuvant, CpG oligodeoxynucleotides, lipopolysaccharide, and polyinosinic polycytidylic acid.

A (pharmaceutical) composition comprising a CD19×CD3 bispecific antibody is, for example, described in WO2007/068354.

Prevention of presence of microorganisms may be ensured both by sterilization procedures and by the inclusion of various antibacterial and antifungal agents, for example, paraben, chlorobutanol, phenol, sorbic acid, and the like. In addition, prolonged absorption of the injectable pharmaceutical form may be brought about by the inclusion of agents which delay absorption, such as aluminum monostearate and gelatin.

The pharmaceutical compositions containing the antibodies comprising a compound of the present invention may also include a suitable salt therefore. Any suitable salt, such as an alkaline earth metal salt in any suitable form (e.g., a buffer salt), may be used in the stabilization of the compound of the present invention. Suitable salts typically include sodium chloride, sodium succinate, sodium sulfate, potassium chloride, magnesium chloride, magnesium sulfate, and calcium chloride. In one embodiment, an aluminum salt is used to stabilize a compound of the present invention in a pharmaceutical composition of the present invention, which aluminum salt also may serve as an adjuvant when such a composition is administered to a patient. The pharmaceutical compositions containing the antibodies may be in a variety of suitable forms. Such forms include, for example, liquid, semisolid and solid dosage forms, such as liquid solutions (e.g., injectable and infusible solutions), dispersions or suspensions, emulsions, microemulsions, gels, creams, granules, powders, tablets, pills, powders, liposomes, dendrimers and other nanoparticles (see for instance Baek et al., Methods Enzymol. 362, 240-9 (2003), Nigavekar et al., Pharm Res. 21(3), 476-83 (2004), microparticles, and suppositories.

A “pharmaceutically acceptable salt” refers to a salt that retains the desired biological activity of the parent compound and does not impart any undesired toxicological effects (see for instance Berge, S. M. et al., J. Pharm. Sci. 66, 1-19 (1977)). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous acids and the like, as well as from nontoxic organic acids, such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Pharmaceutically acceptable carriers include any and all suitable solvents, dispersion media, coatings, antibacterial and antifungal agents, isotonicity agents, antioxidants and absorption delaying agents, and the like that are physiologically compatible with a compound of the present invention. Examples of suitable aqueous and nonaqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

The term “administering” in all of its grammatical forms means administration of a CD19×CD3 bispecific antibody (in the form of a pharmaceutical composition) either as the sole therapeutic agent or in combination with another therapeutic agent.

Accordingly, in the context of the present invention “administration of a CD19×CD3 bispecific antibody” or “administering a CD19×CD3 bispecific antibody” or any other grammatical form thereof means that the CD19×CD3 antibody is in the form of a composition, preferably a pharmaceutical composition, optionally comprising a pharmaceutically acceptable carrier. Accordingly, it is to be understood that a composition, preferably a pharmaceutical composition, comprising a CD19×CD3 bispecific antibody is administered to a human patient. When administered to a patient, preferably a therapeutically effective dose of a composition comprising a CD19×CD3 bispecific antibody is administered to a patient. Accordingly, the composition of the present invention preferably includes a “therapeutically effective amount” of CD19×CD3 bispecific antibody.

Similarly, it is also preferred that the composition comprising a CD19×CD3 bispecific antibody has anti-tumor activity. By “anti-tumor activity” is intended a reduction in the rate of malignant CD19-expressing cell proliferation or accumulation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Therapy with at least one CD19×CD3 bispecific antibody causes a physiological response that is beneficial with respect to treatment of disease states.

The treatment regimen of the present invention comprises administering to said patient a composition comprising a CD19×CD3 bispecific antibody in at least one treatment cycle, wherein the treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time.

When “a first dose of 15 μg/m²/d of the CD19×CD3 bispecific antibody” or “a second dose of 60 μg/m²/d of the CD19×CD3 bispecific antibody” is administered for treatment of Burkitt lymphoma or leukemia, it means that a first or a second dose of the (pharmaceutical) composition comprising a CD19×CD3 bispecific antibody is administered to said patient according to the present invention.

“m²” denotes a square meter of a patient's body surface (BSA). The “normal” average BSA is generally taken to be about 1.73 m² for an adult, for a neonate it is about 0.25 m², for a 2 year old child it is about 0.5 m², for a 9 year old child it is about 1.07 m², for a 10 year old child it is about 1.14 m², for a 12-13 year old child it is about 1.33 m², for men it is about 1.9 m² and for women it is about 1.6 m². However, the BSA can also be calculated more precisely by one of the following formulas (each of these formulas can be applied when calculating the BSA): The Mosteller formula (Mosteller, N Engl J Med 1987 Oct. 22; 317(17): 1098):

BSA (m²)=([Height(cm)×Weight(kg)]/3600)^{1/2} or in inches and pounds:

BSA (m²)=([Height(in)×Weight(lbs)]/3131)^{1/2}

The DuBois formula (DuBois, Arch Int Med 1916 17:863-871):

BSA (m²)=0.007184×Height(cm)^{0.725}×Weight(kg)^{0.425}

The Haycock formula (Haycock, The Journal of Pediatrics 1978 93:1: 62-66):

BSA (m²)=0.024265×Height(cm)^{0.3964}×Weight(kg)^{0.5375}

The Gehan formula (Gehan, Cancer Chemother Rep 1970 54:225-35):

BSA (m²)=0.0235×Height(cm)^{0.42246}×Weight(kg)^{0.51456}

The Boyd formula (Boyd, University of Minnesota Press, 1935)

BSA (m²)=0.0003207×Height(cm)^{0.3}×Weight(grams)^{(0.7285-(0.0188×log 10(grams)}

It is generally preferred that each of the doses disclosed herein can be converted from amount (in μg)/m²/d into μg/d. Accordingly, each of the doses disclosed herein can be applied in the methods/means and uses. For example, a dose of 5 μg/m²/d is converted into 9 μg/d, a dose of 15 μg/m²/d is converted into 28 μg/d and a dose of 60 μg/m²/d is converted into 112 μg/d. According to the present invention, a first dose of 15 μg/m²/d always refers to 28 μg/d and a second dose of 60 μg/m²/d always refers to 112 μg/d when applied in the methods/means and uses of the present invention. It is preferred that a decimal digit that results from the multiplication is either rounded up or rounded down, respectively, to a whole number. For example, a dose of 9.5 μg/d can be rounded down to 9 μg/d and a dose of 28.5 μg/d can be rounded down to 28 μg/d. Likewise, a dose of 9.5 μg/d can be rounded up to 10 μg/d and a dose of 28.5 μg/d can be rounded up to 29 μg/d.

The term “μg” includes “μg of the CD19×CD3 bispecific antibody preparation”. It is preferred that not more than 10% of said CD19×CD3 bispecific antibody preparation is incorrectly folded. It follows that in a preferred embodiment, 90%, 91%, 92%, 93%, 94% or even 95% of the CD19×CD3 bispecific antibody is correctly folded, see, for example, WO 2005/052004 It is also conceivable that the antibody preparation may optionally comprise further ingredients, for example a lyoprotectant, a surfactant, a filler, a binder, and/or bulking agent etc. The amount of such further ingredients is, preferably, not included in the term “μg” as used in the context of the “dose” and/or methods (dosage regimens) of the present invention.

A dose of, for example, 15 μg/m²/d means that 15 μg of the CD19×CD3 bispecific antibody is administered evenly or continuously across one day per square meter body surface of the respective patient. “Continuously across one day” refers to an infusion which is allowed to proceed permanently without interruption.

The term “exceeds” means that the second period of time is at least one day longer than the first period of time. The duration of the second period of time may be variable in view of, for example, the age, sex, body weight, etc. of the human patient. Accordingly, with regard to the methods and means of the present invention said second period of time, no matter whether it refers to the first, second or third treatment cycle or any further treatment cycle, is at least 18 days long, whereby even longer periods of time of for example 19, 20, 25, 30, 35, 40, 45, 50, 55, 60, 65, 70, 75, 80, 81, 82, 83, 84, 85, 86, 87, 88 or 90 days are not excluded. “Longer” is thereby not limited to a (one) complete day as the lowest time unit, i.e. ½ days, or fully hours are also conceivable. It is however preferred that the smallest time unit is one full day. More preferably, said second period of time, no matter whether it refers to the first, second or third treatment cycle or any further treatment cycle, is between 18 days and 60 days, such as 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 days, preferably between 18 days and 30 days, with 24 days being particularly preferred. Thus, according to the present invention the at least one treatment cycle most preferably is 28 days with a first period of time of 4 days and a second period of time of 24 days being preferred.

As used herein, a time interval which is defined as “X to Y” equates with a time interval which is defined as “between X and Y”. Both time intervals specifically include the upper limit and also the lower limit. This means that for example a time interval “18 to 60 days” or between “18 to 60 days” includes a period of time of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43 days.

It is further preferred that the methods and means of the present invention further comprise administering after the first treatment cycle at least a second treatment cycle. Thus, according to the present invention the at least second treatment cycle preferably also comprises 28 days, with a first period of time of 4 days and a second period of time of 24 days being preferred.

Thus, it is preferred that a patient receives at least two treatment cycles of administering to said patient a composition comprising a CD19×CD3 bispecific antibody, wherein the second treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time. The two treatment cycles as described elsewhere herein being preferred refer to an induction cycle of remission according to the present invention.

Preferably, when at least a second treatment cycle is performed, wherein the second treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time, the second period of time exceeds 18 days as described elsewhere herein.

Even more preferably, when at least a second treatment cycle is performed, wherein the second treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time, the second period of time is between 18 and 60 days, preferably between 18 and 30 days, with 24 days being particularly preferred as described elsewhere herein.

According to the present invention in another preferred embodiment, the second treatment cycle is initiated at least 5 days, at least 6 days, at least 7 days, at least 8 days, at least 9 days, at least 10 days, at least 11 days, or at least 12 days, preferably about 5, 6, 7, 8, 9, 10, 11 or 12 days, more preferably 10 days after completing the first treatment cycle.

The term “initiate” in all of its grammatical forms means to start something. Thus, the second treatment cycle is started at least 5 days, at least 6 days, at least 7 days, at least 8 days, at least 9 days, at least 10 days, at least 11 days, or at least 12 days, preferably about 5, 6, 7, 8, 9, 10, 11 or 12 days, more preferably 10 days after completing the first treatment cycle. In other words, after the first treatment cycle is finished a phase of non-treatment follows. After said non-treatment phase, the second treatment cycle as described elsewhere herein is started on the 5^{th} day or later, the 6^{th} day or later, the 7^{th} day or later, the 8^{th} day or later, the 9^{th} day or later, the 10^{th} day or later, the 11^{th} day or later, or the 12^{th} day or later, preferably on the 10^{th} day after having finished the first treatment cycle.

It is further preferred with regard to the methods and means of the present invention that after the first and second treatment cycle at least a third treatment cycle, such as another third, another fourth, another fifth, another sixth, another seventh treatment cycle or even more treatment cycles, preferably comprising the same treatment regimen as used for the first and the second treatment cycle as defined elsewhere herein, is/are administered to said patient. Thus, according to the present invention the at least third treatment cycle, such as another third, another fourth, another fifth, another sixth, another seventh treatment cycle or even more treatment cycles preferably comprises 28 days, with a first period of time of 4 days and a second period of time of 24 days being preferred. Also comprised herein is that the additional treatment cycles, preferably five additional cycles as defined elsewhere herein, may vary with regard to the treatment regimen in that each of these additional cycles, preferably five additional cycles, start with the target dose of 60 μg/m²/d of the CD19×CD3 bispecific antibody right away. Thus, said additional treatment cycles, preferably five additional cycles as defined elsewhere herein, may then comprise administering to said patient a composition comprising a CD19×CD3 bispecific antibody in a first dose of 60 μg/m²/d of the CD19×CD3 bispecific antibody for a period of time, preferably for 28 days in total.

Even more preferred is that after the first and second treatment cycle another five additional treatment cycles, preferably comprising the same treatment regimen as used for the first and the second treatment cycle or alternatively comprising administering to said patient in need thereof the target dose of 60 μg/m²/d of the CD19×CD3 bispecific antibody right away as defined elsewhere herein, are administered to said patient of the present invention.

Each of the five treatment cycles as described elsewhere herein preferably comprises 28 days, with a first period of time of 4 days and a second period of time of 24 days being preferred. The treatment-free interval between the additional 5 cycles can be up to 3 months. This also accounts for the treatment-free interval between the 2^{nd} and the 3^{rd} cycle.

In total the treatment regimen of the methods and means of the present invention comprising preferably five additional treatment cycles is 24 months long. Preferably, a patient is treated with two treatment cycles of the composition comprising a CD19×CD3 bispecific antibody as described elsewhere herein within 66 days for induction of remission. After completing the two treatment cycles within 14 days a response assessment may be performed. In this context, the term “response assessment” refers to assessing the rate of complete hematologic remission in case of Burkitt leukemia and the rate of complete metabolic response (CMR) or partial metabolic response (PMR) by Lugano 2014 criteria in case of Burkitt lymphoma. To verify such so called “primary endpoints” PET/CT scan may be applied in patients with Burkitt lymphoma. In addition for patients with Burkitt leukemia CR has to be documented in the bone marrow by cytology and/or histology. A pretreatment PET/CT scan baseline may be applied during screening, then after first and after the second cycle of Blinatumomab for response assessment. In Burkitt lymphoma patients with an Overall Response Rate (ORR) may receive after 3 months a CT-scan for the next 2 years. For patients with Burkitt leukemia, bone marrow assessment will be performed every 3 months in addition to CT scans during follow up for 2 years.

The term “complete hematological remission” or “complete hematological response” as used herein is to be understood as having no evidence of Burkitt leukemia after standard treatment, e.g. after chemotherapy and/or transplantation. This means that the bone marrow contains fewer than 5% blast cells as determined by light microscopy, the blood cell counts are within normal limits, and there are no signs or symptoms of Burkitt leukemia. Nevertheless, it may occur that not all leukemia cells could be eliminated from the body. Such a patient, though staged as being in remission or complete hematological remission, is still MRD positive, which will be defined in more detail elsewhere herein. These remaining tumor cells may give rise to recurrent leukemia. The means and methods of the invention can be used to kill these remaining tumor cells in order to prevent recurrence of the leukemia originating from the occult leukemia cells remaining in the body after primary therapy. In this way, the means and methods help to prevent disease relapse in Burkitt leukemia patients.

In contrast, a “complete molecular remission” or “complete molecular response” means that there is no evidence of leukemia cells in biopsies of the bone marrow, even when using very sensitive tests such as PCR or FACS analysis. Put in other words: If no MRD, which will be be defined in more detail elsewhere herein, is detectable (<10⁻⁴, i.e. less than one leukemia cell per 10⁴ bone marrow cells), a complete molecular remission is reached.

The incorporation of the Lugano 2014 Criteria for the assessment of lymphoma patients' response to therapy represents an important paradigm shift in the initial and on-study imaging evaluations. The goal of the Lugano 2014 guidelines was to revise the Cheson 2007 criteria, in order to reduce ambiguity and achieve more consistent therapeutic response assessments for patients enrolled in clinical trials evaluating treatment for lymphoma. The most significant aspects of the new guidelines pertain to three major components:
the predominant use of fluorodeoxyglucose (FDG) positron emission tomography (PET) imaging combined with high quality computed tomography (CT) imaging (short: FDG-PET/CT) in the assessment of FDG-avid lymphoma, while CT remains the designated standard for assessment of non-FDG-avid lymphomas;
the replacement of the dichotomous evaluation of FDG uptake (positive vs negative) with a 5-point scale (5PS) assessment for interim and EOT analyses (see Examples)
the premise that all FDG-avid disease (for applicable lymphomatous indications) present in the individual patient is included in each time point (TP) analysis.
   Other updates in the Lugano 2014 criteria include:
the discontinuation of routine BM biopsies in HL and FDG-avid NHL;
the modification of the Ann Arbor staging method;
the recommendation to reduce the total number of routine follow-up surveillance scan procedures.

An on-study Lugano 5PS score of 1, 2 or 3 (see Table 1) as described in the Examples is considered as a complete metabolic response (CMR) (see Table 2) by Lugano 2014 criteria as described elsewhere herein. A score of 4 or 5 (see Table 1) with a decrease of >25% in SUVₘₐₓ (quantitative standardized uptake value (SUV) measurement, which represents the maximum SUV (SUVₘₐₓ), is considered to be a significant decline in FDG uptake, representative of a partial metabolic response (PMR) (see Table 2). A score of 4 or 5 (see Table 1) with an increase of >50% is considered a significant increase in FDG uptake, representative of progressive metabolic disease (PMD), and a change metric between ≤25% decrease and 50% increase in FDG uptake is considered to be no significant change in FDG uptake, representative of no metabolic response (NMR) (see Table 2).

The preferably five additional treatment cycles being administered after the first and the second treatment cycle to the patient in need thereof refer to consolidation or maintenance cycles. Consolidation cycles (also called maintenance cycles) refer to a treatment administered to the patient in need thereof, where a complete response (CR) has been determined as response assessment after the first and the second treatment cycle.

As used herein, a “CD19×CD3 bispecific antibody” (including a CD19×CD3 bispecific single chain antibody) denotes a single polypeptide chain comprising two binding domains. Such CD19×CD3 bispecific single chain antibodies are preferred in the context of the methods/dosage regimen of the present invention. Each binding domain comprises at least one variable region from an antibody heavy chain (“VH or H region”), wherein the VH region of the first binding domain specifically binds to the CD3 epsilon molecule, and the VH region of the second binding domain specifically binds to CD19. The two binding domains are optionally linked to one another by a short polypeptide spacer. A non-limiting example for a polypeptide spacer is Gly-Gly-Gly-Gly-Ser (G-G-G-G-S) and repeats thereof as depicted in SEQ ID NO. 23. Each binding domain may additionally comprise one variable region from an antibody light chain (“VL or L region”), the VH region and VL region within each of the first and second binding domains being linked to one another via a polypeptide linker, for example of the type disclosed and claimed in EP 623679 B1, but in any case long enough to allow the VH region and VL region of the first binding domain and the VH region and VL region of the second binding domain to pair with one another such that, together, they are able to specifically bind to the respective first and second binding domains. Such CD19CD3 bispecific single chain antibodies are described in great detail in WO99/54440 and WO2004/106381 and WO2008/119565.

The term “binding domain” characterizes in connection with the present invention a domain of a polypeptide which specifically binds to/interacts with a given target structure/antigen/epitope. Thus, the binding domain is an “antigen-interaction-site”. The term “antigen-interaction-site” defines, in accordance with the present invention, a motif of a polypeptide, which is able to specifically interact with a specific antigen or a specific group of antigens, e.g. the identical antigen in different species. Said binding/interaction is also understood to define a “specific recognition”. The term “specifically recognizing” means in accordance with this invention that the antibody molecule is capable of specifically interacting with and/or binding to at least two, preferably at least three, more preferably at least four amino acids of an antigen, e.g. the human CD3 antigen as defined herein. Such binding may be exemplified by the specificity of a “lock- and-key-principle”. Thus, specific motifs in the amino acid sequence of the binding domain and the antigen bind to each other as a result of their primary, secondary or tertiary structure as well as the result of secondary modifications of said structure. The specific interaction of the antigen-interaction-site with its specific antigen may result as well in a simple binding of said site to the antigen. Moreover, the specific interaction of the binding domain/antigen-interaction-site with its specific antigen may alternatively result in the initiation of a signal, e.g. due to the induction of a change of the conformation of the antigen, an oligomerization of the antigen, etc. A preferred example of a binding domain in line with the present invention is an antibody. The binding domain may be a monoclonal or polyclonal antibody or derived from a monoclonal or polyclonal antibody.

The term “antibody” comprises derivatives or functional fragments thereof which still retain the binding specificity. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999. The term “antibody” also comprises immunoglobulins (Ig's) of different classes (i.e. IgA, IgG, IgM, IgD and IgE) and subclasses (such as IgG1, IgG2 etc.).

The definition of the term “antibody” also includes embodiments such as chimeric, single chain and humanized antibodies, as well as antibody fragments, like, inter alia, Fab fragments. Antibody fragments or derivatives further comprise F(ab′)2, Fv, scFv fragments or single domain antibodies, single variable domain antibodies or immunoglobulin single variable domain comprising merely one variable domain, which might be VH or VL, that specifically bind to an antigen or epitope independently of other V regions or domains; see, for example, Harlow and Lane (1988) and (1999), cited above. Such immunoglobulin single variable domain encompasses not only an isolated antibody single variable domain polypeptide, but also larger polypeptides that comprise one or more monomers of an antibody single variable domain polypeptide sequence.

As used herein, CD3 epsilon denotes a molecule expressed as part of the T cell receptor and has the meaning as typically ascribed to it in the prior art. In human, it encompasses in individual or independently combined form all known CD3 subunits, for example CD3 epsilon, CD3 delta, CD3 gamma, CD3 zeta, CD3 alpha and CD3 beta. The human CD3 epsilon is indicated in GenBank Accession No.NM_000733.

The human CD19 protein is indicated in GenBank Accession No. AAA69966.

Preferably, the bispecific antibody applied in the methods/dosage regimens of the present invention has the domain arrangement VL(CD19)-VH(CD19)-VH(CD3)-VL(CD3).

It is, however, also envisaged that the CD19×CD3 bispecific antibody as used in the methods of the invention is a CD19×CD3 bispecific single chain antibody. Such CD19×CD3 bispecific single chain antibody could have other domain arrangements, such as

### VH(CD19)-VL(CD19)-VH(CD3)-VL(CD3),

### VL(CD19)-VH(CD19)-VL(CD3)-VH(CD3),

### VH(CD19)-VL(CD19)-VL(CD3)-VH(CD3),

### VL(CD3)-VH(CD3)-VH(CD19)-VL(CD19),

### VH(CD3)-VL(CD3)-VH(CD19)-VL(CD19),

### VL(CD3)-VH(CD3)-VL(CD19)-VH(CD19), or

### VH(CD3)-VL(CD3)-VL(CD19)-VH(CD19).

A preferred CD19×CD3 bispecific antibody applied in the methods of the present invention comprises the
(a) anti-CD3 CDRs of the heavy chain shown as CD3 CDR-H1 in SEQ ID NO: 11 (GYTFTRYTMH), CD3 CDR-H2 in SEQ ID NO: 12 (YINPSRGYTNYNQKFKD) and CD3 CDR-H3 in SEQ ID NO: 13 (YYDDHYCLDY); and/or
(b) anti-CD3 CDRs of the light chain shown as CD3 CDR-L1 in SEQ ID NO: 14 (RASSSVSYMN), CD3 CDR-L2 in SEQ ID NO: 15 (DTSKVAS) and CD3 CDR-L3 in SEQ ID NO: 16 (QQWSSNPLT); and/or
(c) anti-CD19 CDRs of the heavy chain shown as CD19 CDR-H1 in SEQ ID NO: 17 (GYAFSSYWMN), CD19 CDR-H2 in SEQ ID NO: 18 (QIWPGDGDTNYNGKFKG) and CD19 CDR-H3 in SEQ ID NO: 19 (RETTTVGRYYYAMDY); and/or
(d) anti-CD19 CDRs of the light chain shown as CD19 CDR-L1 in SEQ ID NO: 20 (KASQSVDYDGDSYLN), CD19 CDR-L2 in SEQ ID NO: 21 (DASNLVS) and CD19 CDR-L3 in SEQ ID NO: 22 (QQSTEDPWT).

It is more preferred that the CD19×CD3 bispecific single-chain antibody applied in the methods of the present invention comprises the CD3 CDRs of the heavy and light chain. Even more preferably, the CD19×CD3 bispecific single-chain antibody applied in the methods of the present invention comprises the CD3 CDRs of the heavy and light chain as well as the CD19 CDRs of the heavy and light chain.

The CDRs referred to herein are in accordance with the Kabat numbering system. The Kabat numbering scheme is a widely adopted standard for numbering the residues of an antibody in a consistent manner (Kabat et al., Sequences of Proteins of Immunological Interest, 1991).

Alternatively, it is preferred that the CD19×CD3 bispecific single-chain antibody applied in the methods of the present invention comprises the
(a) CD19 variable heavy chain shown in SEQ ID NO: 3 (nucleotide sequence is shown in SEQ ID NO: 4); and/or
(b) CD19 variable light chain shown in SEQ ID NO: 5 (nucleotide sequence is shown in SEQ ID NO: 6); and/or
(c) CD3 variable heavy chain shown in SEQ ID NO: 7 (nucleotide sequence is shown in SEQ ID NO: 8); and/or
(d) CD3 variable light chain shown in SEQ ID NO: 9 (nucleotide sequence is shown in SEQ ID NO: 10).

More preferably, the CD19×CD3 bispecific single-chain antibody applied in the methods of the present invention comprises the CD19 variable heavy and light chain and/or the CD3 variable heavy and light chain. Even more preferably, the CD19×CD3 bispecific single-chain antibody applied in the methods of the present invention comprises the CD19 variable heavy and light chain as well as the CD3 variable heavy and light chain.

In another alternative, it is also preferred that the CD19×CD3 bispecific single-chain antibody comprises an amino acid sequence selected from the group consisting of
(a) an amino acid sequence as depicted in SEQ ID NO: 1;
(b) an amino acid sequence encoded by a nucleic acid sequence as shown in SEQ ID NO: 2;
(c) an amino acid sequence encoded by a nucleic acid sequence having at least 70%, 80%, 90%, 95% or 99% identity to a nucleic acid sequence of (b), wherein said amino acid sequence is capable of specifically binding to CD3 and CD19; and
(d) an amino acid sequence encoded by a nucleic acid sequence which is degenerate as a result of the genetic code to a nucleotide sequence of (b), wherein said amino acid sequence is capable of specifically binding to CD3 and CD19.

It is to be understood that the sequence identity is determined over the entire amino acid sequence. For sequence alignments, for example, the programs Gap or BestFit can be used (Needleman and Wunsch J. Mol. Biol. 48 (1970), 443-453; Smith and Waterman, Adv. Appl. Math 2 (1981), 482-489), which is contained in the GCG software package (Genetics Computer Group, 575 Science Drive, Madison, Wis., USA 53711 (1991). It is a routine method for those skilled in the art to determine and identify an amino acid sequence having e.g. 70%, 80%, 90%, 95%, 96%, 97%, 98% or 99% sequence identity to the amino acid sequences of the CD19×CD3 bispecific antibody described herein (preferably MT103). For example, according to Crick's Wobble hypothesis, the 5′ base on the anti-codon is not as spatially confined as the other two bases, and could thus have non-standard base pairing. Put in other words: the third position in a codon triplet may vary so that two triplets which differ in this third position may encode the same amino acid residue. Said hypothesis is well known to the person skilled in the art (see e.g. http://en.wikipedia.org/wiki/Wobble_Hypothesis; Crick, J Mol Biol 19 (1966): 548-55). It is furthermore a routine procedure for those skilled in the art to determine cytotoxic activity of such an amino acid sequence having e.g. 70%, 80%, 90%, 95%, 96%, 97%, 98% or 99% sequence identity to the nucleotide or amino acid sequences of the CD19×CD3 bispecific single chain antibody described herein. Cytotoxic activity of the CD19×CD3 bispecific single chain antibody or an antibody construct having e.g. 70%, 80%, 90%, 95%, 96%, 97%, 98% or 99% sequence identity to the amino acid sequences of the CD19×CD3 bispecific single chain antibody can be determined by methods as illustrated e.g. in WO 99/54440.

It is particularly preferred, that said CD19×CD3 bispecific single-chain antibody has the amino acid sequence shown in SEQ ID NO: 1. Also particularly preferred is the CD19×CD3 bispecific single-chain antibody as described in WO99/54440 as well as those CD19×CD3 bispecific single-chain antibodies described in WO2004/106381 and WO2008/119565.

Most preferably, said CD19×CD3 bispecific antibody is MT103/AMG103 (Blinatumab) as described in WO99/54440. Therefore, said preferred a CD19×CD3 bispecific (single chain) antibody is also called Blinatumomab. Blinatumomab is a B cell malignancy—directed, recombinant bispecific single-chain CD19×CD3 antibody that binds to CD19 on the surface of almost all B cells and B tumor cells and concomitantly can engage a T cell, thereby triggering the T-cell to kill the target B cell or B tumor cell. Blinatumomab consists of four immunoglobulin variable domains assembled into a single polypeptide chain. Two of the variable domains form the binding site for CD19, a cell surface antigen expressed on most B cells and B tumor cells. The other two variable domains form the binding site for the CD3 complex on T cells. Blinatumomab is designed to direct the body's cytotoxic, or cell-destroying, T cells against tumor cells, and represent a new therapeutic approach to cancer therapy. Blinatumomab is presently in clinical trials.

It also preferred that the bispecific single-chain antibody that is applied in the context of the present invention has an N- and/or C-terminal tag, preferably a C-terminal tag. A preferred example of a C-terminal tag is a His-tag. Said His-tag comprises or consists of six histidine residues in length. It is even more preferred that said His-tag is six histidine residues in length and is located at the C-terminus of the CD19×CD3 bispecific single-chain antibody of the present invention. Thus, in a particularly preferred embodiment of the present invention, said CD19×CD3 bispecific single-chain antibody comprises or consists of a polypeptide as represented by SEQ ID NO: 1 and additionally of a hexa-histidine-tag (HHHHHH) which is located at its C-terminus. It is also preferred that the protein purification tag (His-tag being more preferred and Hexa-His-tag being most preferred) is linked to the C-terminus of said CD19×CD3 bispecific single-chain antibody of the present invention (preferably consisting of or comprising SEQ ID NO: 1) via a peptide bond.

In a further preferred embodiment, said CD19×CD3 bispecific single-chain antibody including the above mentioned protein purification tag(s), His-tags being preferred and Hexa-His-tags at the C-terminus being more preferred, is produced in a host cell as defined herein. CHO is thereby a particularly preferred host cell.

The uninterrupted administration of the CD19×CD3 bispecific antibody may be intravenous, parenteral, subcutaneous, transdermal, intraperitoneal, intramuscular or pulmonary. The intravenous mode of administration will in most cases be the mode of choice for uninterruptedly administering the CD19×CD3 bispecific single chain antibody and, as the case may be, for co-administration of a pharmaceutical agent as part of a regimen of co-therapy. As such, intraveneous administration is especially preferred. In this case, a suitable metering device such as the multi-therapy infusion pump model 6060 manufactured by Baxter may advantageously be chosen. Whatever metering device is chosen, it should be of such design and construction as to minimize or, better, preclude an interruption of administration of therapeutic agent in the event of cartridge exchange and/or power cell replacement or recharging. This may be accomplished, for example by choosing a device with a secondary reservoir of CD19×CD3 bispecific single chain antibody solution apart from the cartridge to be exchanged so that continuous infusion from this secondary reservoir into the pediatric patient may continue even while the empty or almost empty cartridge is removed and replaced with a fresh one.

A mode of intraveneous administration and, as the case may be, of co-administration as part of a regimen of co-therapy involves the implantation of a pump into the body of the pediatric patient for metering such administration. One of ordinary skill in the art is aware of such metering pumps, for example model 6060 manufactured by Baxter as set forth above.

Most preferably, the route of administration of the first dose of the CD19×CD3 bispecific antibody for a first period of time of 4 days as defined elsewhere herein and the second dose of the CD19×CD3 bispecific antibody for a second period of time as defined elsewhere herein referred to herein is an intravenous administration. Even more preferably the route of administration being intravenous is performed continually (continuously).

A continual/continuous administration refers to an administration which is essentially without interruption. “Essentially without interruption” includes a continual administration usually without an uninterrupted flow or spatial extension.

In a preferred embodiment the method of the present invention further comprises administering pentosane polysulfate (PPS) prior to the first administration of the first dose and prior to the first administration of the second dose of the CD19×CD3 bispecific antibody of each treatment cycle to said patient. This means that PPS according to the present invention is given prior to the first administration of the first dose of the CD19×CD3 bispecific antibody as defined elsewhere herein and prior to the first administration of any dose step. Any dose step as used herein refers to any increase of the dose of the CD19×CD3 bispecific antibody, which according to the present invention refers to the second dose of the CD19×CD3 bispecific antibody as it is defined elsewhere herein.

First data have shown that PPS can prevent treatment interruption because of neurologic (neurological) adverse events (AEs) as will be defined elsewhere herein. Although the number of patients per group was small, intrapatient single-stepwise dose escalation to 60 mg/m²/day (112 μg per day) plus PPS was the most promising, because even no treatment discontinuations due to neurologic AEs were necessary (Goebeler et al. J Clin Oncol. 2016).

Thus, the present inventors found that a co-medication scheme of the heparinoid pentosanpolysulfate (PPS), a small-molecule inhibitor of P-selectin being administered prior to and/or concomitantly with Blinatumomab (Höpfner et al. J Pharm Pharmacol. 2003; 55:697-706) is advantageous for preventing neurological adverse effects as described elsewhere herein such as neurological toxicity. P-selectin is known to mediate the first step of leukocyte adhesion to endothelial cells and seems to play a particular important role for the extravasation of circulating leukocytes via meningeal microvessels to the leptomeningeal space and the meninges (Kivisäkk et al. Proc Natl Acad Sci USA. 2003; 100:8389-94).

Thus, according to the present invention, in another aspect, PPS for use in a method for amelioration, treatment and/or prophylaxis of neurological adverse events in Burkitt lymphoma or leukemia caused by the administration of a CD19×CD3 bispecific antibody as described elsewhere herein is also comprised herein, wherein PPS is administered prior to the first dose and prior to the second dose of the CD19×CD3 bispecific antibody. According to the present invention, PPS as it is used in this particular aspect, may be administered as it is defined elsewhere herein for the method of the present invention.

In this context, the term “amelioration” as used herein is synonymous with improvement. If a Burkitt lymphoma or leukemia patient's condition having neurological AEs which may result from the treatment of said patient in need thereof with the CD19×CD3 bispecific antibody of the present invention, shows amelioration, the patient is clearly better—there is some improvement in her or his clinical condition with regard to the neurological AEs.

In this context, the term “treatment” is defined as the application or administration of PPS to a patient, or application or administration of PPS to an isolated tissue or cell line from a patient, where the patient suffers from neurological adverse events in Burkitt lymphoma or leukemia, is at a risk of developing neurological adverse events in Burkitt lymphoma or leukemia, has a symptom of neurological adverse events in Burkitt lymphoma or leukemia, or a predisposition towards neurological adverse events in Burkitt lymphoma or leukemia, where the purpose is to cure, heal, alleviate, relieve, alter, remedy, ameliorate, improve, or affect neurological adverse events in Burkitt lymphoma or leukemia, the symptoms of neurological adverse events in Burkitt lymphoma or leukemia, or the predisposition towards neurological adverse events in Burkitt lymphoma or leukemia.

The term “prophylaxis” as used herein refers to an interventional measure, method and/or a procedure to prevent or obviate the development of future neurological adverse events in Burkitt lymphoma or leukemia caused by the administration of the CD19×CD3 bispecific antibody as described elsewhere through the administration of PPS as defined elsewhere herein. The term “prevention” may also be used interchangeably with the term “prophylaxis” herein.

When the term “caused by” is used in the context with the administration of said antibody of the present invention, it refers to resulting from the treatment of said patient (in need thereof) with said antibody. Herein, said patient is subject to therapy with said antibody of the present invention.

In one embodiment, neurological adverse events may be comprised by clinical adverse events. The term “clinical adverse events” used herein caused by the administration of the CD19×CD3 bispecific antibody as described elsewhere in a patient thus comprises in particular neurological adverse events. Said neurological adverse event, which sometimes is also denoted as “neurological symptom” or “neurological adverse effect” or “central nervous system adverse event (CNS AE)”, includes but is not limited to conditions of a human patient such as all forms of pain, headache, muscle weakness/incoordination, balance disorder, speech disorder/impairment, sensual disturbance/abnormalities, dizziness, ataxia, apraxia, tremor, aphasia, dysphasia, confusion, disorientation, hallucination, cerebellar symptoms, encephalopathy, seizure, (grand mal) convulsion. Specifically, neurological reactions observed during treatment with a CD3 binding domain include for example confusion and disorientation. “Confusion” as used herein refers to loss of orientation which is the ability to place oneself correctly in the world by time, location, and personal identity, and often memory which is the ability to correctly recall previous events or learn new material. The patients usually have difficulties to concentrate and thinking is not only blurred and unclear but often significantly slowed down. Patients with neurological reactions also suffer from loss of memory. Frequently, the confusion leads to the loss of ability to recognize people and/or places, or to tell time and the date. Feelings of disorientation are common in confusion, and the decision-making ability is impaired. Neurological reactions further comprise blurred speech and/or word finding difficulties. This disorder may impair both, the expression and understanding of language as well as reading and writing. Besides urinary incontinence, also vertigo and dizziness may accompany neurological reactions in some patients.

Pentosanpolysulfate (PPS; C₁₄H₂₆O₂₁S₄), also known as Pentosan Polysulphate, Xylan Hydrogen Sulfate, Xylan Polysulfate, is a semi-synthetically produced heparin-like macromolecular carbohydrate derivative, which chemically and structurally resembles glucosaminoglycans. It is a white odorless powder, slightly hygroscopic and soluble in water to 50% at pH 6. It has a molecular weight of 4000 to 6000 Dalton. A particular preferred PPS that was administered to a patient mentioned in the present invention is pentosanpolysulfate SP54® commonly known and available in the market (e.g. from bene Arzneimittel GmbH).

PPS is, for example, sold under the name Elmiron® by Ortho-McNeil Pharmaceutical, Inc. and is thus far the only oral medication approved by the U.S. FDA for the treatment of interstitial cystitis, also known as painful bladder syndrome. For treatment of this condition, PPS is administered orally, however, it can alternatively be administered intravenously.

The term “pentosanpolysulfate (or PPS)” encompasses semi-synthetically produced heparin-like macromolecular carbohydrate derivatives.

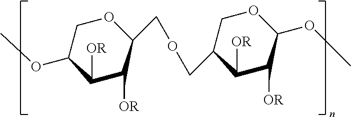

In the sense of the present invention, pentosanpolysulfate is a mixture of linear polymers of β1→4-linked xylose, usually sulfated at the 2- and 3-positions and occasionally substituted at the 2-position with 4-O-methyl-α-D-glucuronic acid-2,3-O-sulfate. Accordingly, PPS may also be designated as β1→4-D-Xylan-2,3-bis(hydrogen sulfate).

By way of example, a semi-synthetically produced heparin-like macromolecular carbohydrate derivative such as in particular PPS is, for example, producible (obtainable) as follows: its polysaccharide backbone, xylan is, for example, extracted from the bark of the beech tree or other plant sources and is then treated with sulfating agents such as chlorosulfonic acid or sulfuryl chloride and acid. After sulfation, PPS is usually treated with sodium hydroxide to yield the sodium salt which is a preferred salt of the present invention. Processes for the production of a semi-synthetically produced heparin-like macromolecular carbohydrate derivative such as in particular PPS are, for example, disclosed in U.S. Pat. No. 2,689,848 or US 2010/0105889.

More specifically, with regard to the methods and means of the present invention, PPS is administered in the range between 24 hours and 2 hours, such as 24, 23, 22, 21, 20, 19, 18, 17, 16, 15, 14, 13, 12, 11, 10, 9, 8, 7, 6, 5, 4, 3 or 2 hours, prior to the first administration of the first dose and prior to the first administration of the second dose of the CD19×CD3 bispecific antibody, no matter whether it is prior to the first administration of the first dose and prior to the first administration of the second dose of the CD19×CD3 bispecific antibody in the at least first treatment cycle, in the at least second treatment cycle, in the at least third treatment cycle or in any further treatment cycle. Most preferably, PPS is administered 24 hours prior to the first administration of the first dose and prior to the first administration of the second dose of the CD19×CD3 bispecific antibody, no matter whether it is prior to the first administration of the first dose and prior to the first administration of the second dose of the CD19×CD3 bispecific antibody in the at least first treatment cycle, in the at least second treatment cycle, in the at least third treatment cycle or in any further treatment cycle.

According to the methods and means of the invention, PPS administration is preferably continued for at least 3 consecutive days after the first administration of the first dose and the second dose of the CD19×CD3 bispecific antibody in each of the at least one treatment cycle as defined elsewhere herein. More preferably, PPS administration is continued for 3 consecutive days (72 hours) after the first administration of the first dose and after the first administration of the second dose of the CD19×CD3 bispecific antibody in each of the at least one treatment cycle as defined elsewhere herein. Having defined this, it means that when PPS is administered prior to the first and prior to the second dose of the CD19×CD3 bispecific antibody, said administration may then further be continued for at least 3 consecutive days, preferably for 72 hours, after administering the first and the second dose of said antibody. When PPS is administered between 24 hours and 2 hours, preferably 24 hours, prior to the first dose and prior to the second dose of the CD19×CD3 bispecific antibody as defined elsewhere herein and said administration is then continued for at least 3 consecutive days, preferably for 72 hours, after administering the first and the second dose of said antibody, this means that preferably the total administration period is 8 days (preferably administration of PPS 24 hours (1 day) before the first dose of said antibody according to the present invention followed by 7 additional days of continuous PPS administration after the administration of the first dose of said antibody).

Accordingly, PPS administration is preferably continued for 7, 8, 9 or 10 consecutive days after the first administration of the first dose of the CD19×CD3 bispecific antibody in each of the at least one treatment cycle as defined elsewhere herein, more preferably PPS administration is continued for 7 consecutive days after the first administration of the first dose of the CD19×CD3 bispecific antibody in each of the at least one treatment cycle as defined elsewhere herein. In this context, the term “consecutive” means following one after another without interruption.

In the context of the present invention, PPS is preferably administered intravenously to a patient, even more preferably orally. Preferably, PPS is administered in the form of a tablet as described elsewhere herein. Typical doses are 100, 150, 200 or 300 mg, administered 1-3 times per day, with a maximum amount of 600 mg/day. Typically, the daily dose of PPS is between 100 and 600 mg such as 100, 150, 200, 250, 300, 350, 400, 450, 500, 550 or 600 mg. For example, 100 mg PPS may be administered 3-6 times a day. Similarly, 200 mg PPS may be administered 2-3 times a day. Alternatively, 300 mg PPS may be administered 2 times a day.

With regard to the methods and means of the invention, PPS is preferably administered in a dose of 300 mg per day. In other words, PPS is preferably administered daily in a dose of 300 mg. More preferably, PPS is administered in a dose of 300 mg per day prior to the first administration of the first dose and prior to the first administration of the second dose of the CD19×CD3 bispecific antibody of the at least one treatment cycle as described elsewhere herein. Even more preferably, PPS is administered in a dose of 300 mg per day 24 hours prior to the the first administration of the first dose and prior to the first administration of the second dose of the CD19×CD3 bispecific antibody of the at least one treatment cycle as described elsewhere herein. Most preferably, PPS is first administered in a dose of 300 mg per day 24 hours prior to the first administration of the first dose and prior to the first administration of the second dose of the CD19×CD3 bispecific antibody of the at least one treatment cycle and then the administration of 300 mg per day PPS is continued for at least 3 consecutive days, preferably for 72 hours, after the first administration of the first dose and after the first administration of the second dose of the CD19×CD3 bispecific antibody in each of the at least one treatment cycle as described elsewhere herein. Accordingly, PPS administration of 300 mg per day is preferably continued for 7, 8, 9, or 10 consecutive days, more preferably for 7 consecutive days after the first administration of the first dose of the CD19×CD3 bispecific antibody.

In the particular preferred embodiment of the present invention, said daily dose of 300 mg of PPS is a dose of 100 mg of PPS administered 3 times per day. In other words, the administration of a dose of 300 mg per day is most preferably three doses of 100 mg per day. In said particular preferred embodiment, 100 mg of PPS to be administered 3 times per day (three doses of 100 mg per day) is administered prior to the first administration of the first dose and prior to the first administration of the second dose of the CD19×CD3 bispecific antibody of the at least one treatment cycle as described elsewhere herein. Preferably, in said particular preferred embodiment 100 mg of PPS to be administered 3 times per day (three doses of 100 mg per day) is administered within 24 hours prior to the first administration of the first dose and within 24 hours prior to the first administration of the second dose of the CD19×CD3 bispecific antibody in each of the at least one treatment cycle as described elsewhere herein. Even more preferably, 100 mg of PPS to be administered 3 times per day (three doses of 100 mg per day) is administered within 24 hours prior to the first administration of the first dose and within 24 hours prior to the first administration of the second dose of the CD19×CD3 bispecific antibody in each of the at least one treatment cycle as described elsewhere herein and then the administration of 100 mg PPS to be administered 3 times per day is continued for at least 3 consecutive days, preferably for 72 hours, after the first administration of the first dose and after the first administration of the second dose of the CD19×CD3 bispecific antibody in each of the at least one treatment cycle as described elsewhere herein. Accordingly, 100 mg PPS to be administered 3 times per day is preferably continued for 7, 8, 9, or 10 consecutive days, more preferably for 7 consecutive days after the first administration of the first dose of the CD19×CD3 bispecific antibody.

In the context of the particular preferred embodiment when PPS is administered as 100 mg 3 times per day (preferably in the form of tablets), the term “within 24 hours” means administration of 100 mg PPS to be administered 3 times a day a) 24 hours prior to the first administration of the first dose and prior to the first administration of the second dose of the CD19×CD3 bispecific antibody in each of the at least one treatment cycle as described elsewhere herein, b) 18 hours prior to the first administration of the first dose and prior to the first administration of the second dose of the CD19×CD3 bispecific antibody in each of the at least one treatment cycle as described elsewhere herein, and c) 12 hours prior to the first administration of the first dose and prior to the first administration of the second dose of the CD19×CD3 bispecific antibody in each of the at least one treatment cycle as described elsewhere herein, or

administration of 100 mg PPS to be administered 3 times a day a) 24 hours prior to the first administration of the first dose and prior to the first administration of the second dose of the CD19×CD3 bispecific antibody in each of the at least one treatment cycle as described elsewhere herein, b) 16 hours prior to the first administration of the first dose and prior to the first administration of the second dose of the CD19×CD3 bispecific antibody in each of the at least one treatment cycle as described elsewhere herein, and c) 8 hours prior to the first administration of the first dose and prior to the first administration of the second dose of the CD19×CD3 bispecific antibody in each of the at least one treatment cycle as described elsewhere herein.

It is within the scope of the methods and means of the invention that the treatment according to the present invention as defined elsewhere herein may result in a MRD negative status of the Burkitt lymphoma or leukemia patient. Preferably, the methods and means of the invention leads to the elimination of all acute leukemia cells from the body of the patient, resulting in an MRD-negative Burkitt lymphoma or leukemia status, as defined herein. Preferably, in the case of a Burkitt lymphoma or leukemia patient of the present invention said complete molecular response is MRD negativity (as defined hereinafter) which decreases the risk of recurrence of Burkitt lymphoma or leukemia.

The term “minimal residual disease (MRD)” as defined herein denotes a term used after treatment e.g. with chemotherapeutics when leukemic cells cannot be found in the bone marrow using standard tests, such as microscopic methods. Rather, more sensitive tests such as flow cytometry (FACS based methods) or polymerase chain reaction (PCR) have to be used in order to find evidence that leukemia cells remained in the bone marrow of the Burkitt lymphoma or leukemia patient. More specifically, the presence of leukemia cells below the cytological detection limit (5% leukemic cells) is defined as minimal residual disease (MRD). If no MRD is detectable (<10⁻⁴, i.e. less than 1 leukemia cell per 10⁴ bone marrow cells detectable), a complete molecular remission is reached (MRD negativity or MRD negative status). An “MRD positive status” as defined herein means a signal measured by PCR or FACS above detection limit or a quantitative threshold. An “MRD negative status” as defined herein means below detection limit and/or below a quantitative threshold measured by PCR or FACS. The MRD status can be measured by PCR or FACS analysis in that the individual cytogenetic abnormalities mentioned herein, and/or rearrangements of immunoglobulin genes or T-cell receptor (TCR) rearrangements are quantitatively detected. For example, PCR analysis can detect fusion transcripts such as t(8;14), t(2;8) or t(8;22) translocations as well as individual clonal rearrangements of immunoglobulins (IgH) and/or T-cell receptor genes (TCR).

Monitoring MRD has several important roles: determining whether treatment has eradicated the disease or whether traces remain, comparing the efficacy of different treatments, monitoring patient remission status and recurrence of the disease and choosing the treatment that will best meet those needs (personalization of treatment).

In another preferred embodiment of the methods and means of the invention, MRD is measured with quantitative detection of at least one of the cytogenetic abnormalities or rearrangements selected from the group consisting of:
t(8;14);
t(2;8;);
t(8;22);
hyperdiploidy or simultaneous trisomies of chromosomes 4, 10, and 17;
hypodiploidy (i.e. less than 44 chromosomes);
rearrangements of immunoglobulin genes; and
T-cell receptor (TCR) rearrangements.

MRD is measured with quantitative detection of at least one of: (i) the individual cytogenetic abnormalities mentioned herein, such as t(8;14); t(2;8;); t(8;22); hyperdiploidy (or simultaneous trisomies of chromosomes 4, 10, and 17), hypodiploidy (i.e. less than 44 chromosomes), or (ii) the rearrangements of immunoglobulin genes or T-cell receptor (TCR) rearrangements. Said quantitative detection of the mentioned cytogenetic abnormalities or rearrangements is preferably carried out by using, for example, PCR or FACS analysis.

Also within the scope of the methods and means of the invention is that the patient being treated may be characterized by a B:T cell ratio of less than 1:8.

A “B:T cell ratio” as used herein refers to the ratio of the number of B cells and the number of T cells. It is preferably determined in a sample taken from a human patient. Preferably, the sample is taken from the peripheral blood of a human patient. The number of B or T cells, for example, in a peripheral blood sample can be determined by any means usually applied in the art, for example, by FACS analysis.

The B:T cell ratio of said patient of the present invention is preferably about 1:8 or lower including a B:T cell ratio of about 1:9, 1:10, 1:11, 1:12, 1:13, 1:14, 1:15, 1:20, 1:100, 1:200, 1:400, 1:500, 1:1000, 1:2000, 1:3000, 1:4000, 1:5000 or even lower, with 1:9 1:10, 1:50, 1:100, 1:500, 1:1000 being preferred, with 1:9 being particularly preferred.

“Determining the B:T cell ratio” includes

(a) determining the total B cell number in a sample from a patient, preferably in a peripheral blood sample of the patient;
(b) determining the total T cell number in sample from a patient, preferably in a peripheral blood sample of the patient;
(c) calculating the ratio of the B cell number of step (a) and the T cell number of step (b) in order to obtain a B:T cell ratio.

Of note, a low B:T cell ratio can also be seen as high T:B ratio; and vice versa. Accordingly, the ratios provided herein for a low B:T cell ratio would then have to be reversed.

In contrast, patients showing a B:T cell ratio higher than about 1:5, including a B:T cell ratio of higher than about 1:4, 1:3, 1:2, 1:1, 2:1, 3:1, 4:1, 5:1, 6:1, 7:1, 8:1, 9:1, 10:1 or higher, have a decreased risk of suffering from potential adverse effects upon administration of a CD19×CD3 bispecific antibody.

However, a B:T cell ratio is an indicator as to whether or not a patient is at a risk of potential adverse side effects. Specifically, a B:T cell ratio about or lower 1:5 is an indicator that patients are at a risk of potential side effects, while a B:T cell ratio higher than about 1:5 is an indicator that patients have no or at least have a decreased risk of potential side effects as mentioned elsewhere herein.

Specifically, the patient, to whom the present treatment is administered, encounter CNS events, if they have a B:T cell ratio of about 1:8 or lower. Such low B:T cell ratio may be established as a potential high risk factor for the occurrence of adverse effects including neurological reactions in the treatment of malignant CD19 positive lymphocytes occurring in leukemias and lymphomas. Accordingly, a B:T cell ratio of about 1:8 or lower is indicative for a risk of about 50% to develop adverse effects.

Thus, the term “indicative for” when used in the context of the method of the first aspect of the present invention means that a patient has an increased risk of about 50% to develop potential adverse effects if the B:T cell ratio is about 1:8 or lower or has a decreased risk of potential adverse effects if the B:T cell ratio is higher than 1:8.

An “adverse effect” is a harmful and undesired effect resulting from medication in the treatment of a patient with a CD19×CD3 bispecific antibody. An adverse effect may also be termed a “side effect”. Some adverse effects only occur only when starting, increasing or discontinuing a treatment. The inventors have observed that the adverse effect seen in the treatment of patients with a CD19×CD3 bispecific antibody occurred after about 12 to 120 hours after the start of the treatment and are reversible.

An adverse effect may cause medical complications. Neurological reactions in patients treated with a CD19×CD3 bispecific antibody have been observed so far. These neurological reaction, unless they can be stopped or avoided, lead to non-compliance with the CD19×CD3 bispecific antibody treatment.

In clinical trials, a general distinction can be made between adverse effects (AEs) and serious adverse effects (SAEs). Specifically, adverse effects can be classified in 5 grades in accordance with the Common Terminology Criteria for Adverse Events (CTCAE). Grade 1 relates to mild AE, Grade 2 to moderate AE, Grade 3 to severe AE, Grade 4 to life-threatening or disabling AE, while Grade 5 means death related to AE.

In accordance with the present invention an adverse effect is preferably characterized by a neurological reaction (also sometimes referred to herein as “CNS reaction” or “CNS event”, for which reason these terms can be equally used). Said neurological reaction is preferably one or more selected from the group consisting of: confusion, ataxia, disorientation, dysphasia, aphasia, speech impairment, cerebellar symptoms, tremor, apraxia, seizure, grand mal convulsion, palsy, and balance disorder.

The degree of an adverse effect can, for example, be measured in accordance with the NCI Common Terminology Criteria for Adverse Events v3.0 (CTCAE) (Publish Date: Dec. 12, 2003) in grades. A Grade refers to the severity of the adverse effects. The CTCAE v3.0 displays grades 1 through 5 with unique clinical descriptions of severity for each adverse effects:

Grade 1: mild adverse effects
Grade 2: Moderate adverse effects
Grade 3: Severe adverse effects
Grade 4: Life-threatening or disabling adverse effects.
Grade 5: Death of the patient

Thus, the patient being treated with the methods and means of the invention, which can be characterized by a B:T cell ratio of less than 1:8, may have adverse effects of grade 5. Therefore, the patient being treated with the methods and means of the invention, which can be characterized by a B:T cell ratio of less than 1:8, refers to a high-risk patient.

A “patient” is a mammal, preferably a primate, most preferably a human being/human individual (or subject). Said patient will be or is treated with a CD19×CD3 bispecific antibody. In accordance with the present invention, the patient is suspected/assumed to comprise or already comprises malignant CD19 positive lymphocytes (in particular B cells). In the latter case, said patient has already been diagnosed (preferably, as described herein above) to comprise such cells and, thus, suffers from Burkitt lymphoma or leukemia. These malignant CD19 positive lymphocytes (in particular B cells) are present in a patient developing and/or suffering from Burkitt lymphoma or leukemia. In accordance with the present invention a patient is thus in need of a treatment of malignant CD19 positive lymphocytes.

A patient having one or more of the following criteria is excluded from the methods and means of the present invention:
1) CNS disease documented by cytological examination
2) HIV infection
3) Autologous or allogeneic HSCT within 90 days
4) Symptoms and/or clinical signs and/or radiological and/or sonographic signs that indicate an acute or uncontrolled chronic infection, any other concurrent disease or medical condition that could be exacerbated by the treatment or would seriously complicate compliance with the protocol,
5) Bilirubin greater than >3.0 mg/dl (unless Gilbert disease is documented)
6) Creatinine Clearance <50 ml/min
7) Clinically relevant CNS pathology requiring treatment (e.g. unstable epilepsy)
8) Active secondary maligancies
9) Inability to read, understand and sign informed consent
10) Cardiac ejection fraction of less than 40%.

If a patient according to the present invention suffers from high tumor burden, preferably lactatdehydrogenase (LDH) ≥500 U/I and/or bone marrow involvement of at least 50%, a prephase treatment may be administered. Such pretreatment may comprise administering to said patient up to 1 g/m² cyclophosamide and optional 500 mg Prednison-H up to 5 days.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. The term “at least one” refers, if not particularly defined differently, to one or more such as two, three, four, five, six, seven, eight, nine, ten or more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

The term “about” means plus or minus 10%, preferably plus or minus 5%, more preferably plus or minus 2%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the following examples, offered for illustrative purposes only. The examples are not intended to limit the scope of the present invention in any way.

### Examples of the Invention

Study Design

This is an open-label phase II trial of Blinatumomab as continuous iv. infusion in patients with relapsed/refractory Burkitts leukemia or lymphoma. Patients receive 2 cycle of 4 days Blinatumomab in the dosage of 28 μg/d followed by 24 days Blinatumomab c.iv. at 112 μg/d (e.g one cycle) as induction and up to 5 additional cycles as consolidation/maintenance.

Patients undergo screening procedures to determine the eligibility within 28 days prior to the initial administration of Blinatumomab.

Study Duration

The treatment period is approximately 24 months. Patients are initially be treated with two cycles Blinatumomab c.iv. within 66 days for induction of remission and response assessment is performed within 14 days after completing two cycles of Blinatumomab. Up to 5 additional cycles as consolidation/maintenance cycles are administered within 24 months in total. The follow up period is 24 months after treatment initiation of the last patient enrolled.

Sample Size

20 patients are required to determine the efficacy of Blinatuomab in patients with relapsed/refractory Burkitt leukemia or Burkitt lymphoma.

Target Population

Patient must have documented CD19 positive active Burkitts leukemia or lymphoma which is either refractory to first salvage, relapsed after first salvage or any patients beyond second salvage with either relapsed or refractory Burkitts leukemia or lymphoma. All patients must have received CD20 directed antibody therapy as part of their previous chemotherapy regiments.

Treatment

Patients receive Blinatumomab continuously intravenously (c.iv.) at the dose of 28 μg/d (first dose) for the first 4 days and for the remaining 24 days at 112 μg/d (second dose) (1 cycle). A second 28 day cycle is initiated within 10 days after completing the first cycle with a run in phase of 28 μg/d (first dose) for 4 days followed by 24 days at 112 μg/d (second dose). Up to 5 additional cycles as consolidation/maintenance cycles may be administered within 24 months in total. Oral Pentosan Polysulfat (PPS) of 300 mg/d (3×100 mg) is given in each treatment cycle 24 hours prior to the first administration of the first dose and prior to the first administration of the second dose of Blinatumomab. Said administration of PPS is then continued for 72 hours after administration of the first dose and the second dose of Blinatumomab, in that the administration period of PPS accounts for 8 days in total (24 hr before blin+72 hours during blin.+24 hr before step+72 hr after step=8 days). In other words, PPS of 300 mg/d (3×100 mg) is given 24 hours prior to the first administration of the first dose of Blinatumomab and is then continued for the first 7 days of Blinatumomab for neurotoxcitiy protection of every cycle of Blinatumomab.

Endpoints

The primary endpoint is the rate of complete hematologic remission in case of Burkitt Leukemia and the rate of complete metabolic response (CMR) by Lugano 2014 criteria (see Table 1 and 2) in case of Burkitt lymphoma within 2 cycles of Blinatumomab induction treatment the Overall Response Rate (ORR) response after two cycles of Blinatumomab.

ORR (CR and PR) is determined by both Lugano and Cheson criteria for Burkitt lymphoma patients. For patients with Burkitt leukemia CR has to be documented in addition by bone marrow biopsy.

The secondary endpoints are
Overall survival
Relapse-free survival
Response duration
Rate and duration of treatment interruptions, dose reductions, mitigation strategies and rate of withdrawals
MRD response within 2 cycles of Blinatumomab induction treatment
Rate and grade of adverse events (AE) according to CTC-AE version 5
Rate of treatment interruptions and treatment discontinuations
   Serum and bone marrow material are stored for further exploratory research.

TABLE 1
Modified Lugano 5-point scale (5PS)

| Score | Description |
|---|---|
| 1 | No uptake |
| 2 | Uptake ≤ mediastinum |
| 3 | Uptake > mediastinum but ≤ liver |
| 4 | Uptake moderately increased |
| | above liver at any site |
| 5 | Markedly increased uptake |
| | above liver at any site |
| NE | Not evaluable |
| X | Any areas of uptake not likely to |
| | be related to lymphoma |

Note:
Data from Cheson et al.¹⁰

TABLE 2
Determination of Lugano PET-based on-study response

| Lesion | Change from | New | Lugano |
|---|---|---|---|
| 5PS | screening | lesions | response |
| 1, 2, or 3 | Decrease | No | CMR |
| 4 or 5 | Decrease | No | PMR |
| 4 or 5 | No change | No | NMR |
| 4 or 5 | Increase | Yes/no | PMD |
| Any | Any | Yes | PMD |

Abbreviations: PET, positron emission tomography;
5PS, 5-point scale;
CMR, complete metabolic response;
PMR, partial metabolic response;
NMR, no metabolic response;
PMD, progressive metabolic disease.

Key Inclusion criteria
1) CD19 positive relapsed or refractory Burkitt's leukemia or lymphoma (based on WHO criteria)
2) Prior CD20 directed antibody therapy in combination with chemotherapy
3) ECOG Performance status 0-2; in patient with ECOG 3 which is due to disease progression may also be included.
4) At least 18 years old
5) Signed informed consent

Key Exclusion criteria
11) CNS disease documented by cytological examination
12) HIV infection
13) Autologous or allogeneic HSCT within 90 days
14) Symptoms and/or clinical signs and/or radiological and/or sonographic signs that indicate an acute or uncontrolled chronic infection, any other concurrent disease or medical condition that could be exacerbated by the treatment or would seriously complicate compliance with the protocol,
15) Bilirubin greater than >3.0 mg/dl (unless Gilbert disease is documented)
16) Creatinine Clearance <50 ml/min
17) Clinically relevant CNS pathology requiring treatment (e.g. unstable epilepsy)
18) Active secondary maligancies
19) Inability to read, understand and sign informed consent
20) Cardiac ejection fraction of less than 40%.

Pretreatment

Patient with high tumor burden e.g. LDH ≥500 U/I and/or bone marrow involvement of at least 50%, a prephase treatment to bedulk the patient with up to 1 g/m² cyclophosamide and optional 500 mg Prednison-H up to 5 days should be administered.

### Example 1

A 30 year old patient with Stage 4 Burkitt Lymphoma had experienced relapsed disease within 6 months after diagnosis. The relapse of the patient's Burkitt Lymphoma turned out to be refractory to any high dose chemotherapy.

A novel regimen of blinatumomab was applied. The initial dose was 28 μg/day instead of 9 μg/day. This dose was escalated to a dose of 112 μg/day after 4 days instead of after 7 days. This was the 1st case, in which the target dose of 112 μg/day was reached so fast, and this has been the fasted escalation to the target dose of 112 μg/day executed to date.

For the 1st time as medication to prevent neurological toxicity an oral formulation of the P-selectin and angiogenesis inhibitor pentosane polysulfate (PPS) was administered. The total dose per day was 300 mg, which were given as 100 mg tablets 3 times per day over 7 days. No symptoms or signs of neurological toxicity of any grade were recorded. The low B:T cell ratio below 1:8 implied a risk of approximately 50% to develop neurological toxicity for the patient at this dose of blinatumomab (see summery of data below from the neurological advisory board on 12 Jan. 2010, Munich, NH Hotel Muenchen Airport).

Incidence of Neurological Events Depends on B:T Cell Ratio

| | Neurological Events Leading to Treatment | |
|---|---|---|
| | Discontinuation/Treatment Cycle | |
| | B:T Cell Ratio in Blood | |
| Dose Level | Low (<1:8) | High (≥1:8) |
| <15 μg/m²/d | 0/3 | 0/10 |
| 15/30 μg/m²/d | 4/8 | 0/15 |
| 60 μg/m²/d | 5/8 | 0/12 |
| 90 μg/m²/d | 1/1 | 2/3 |

Based on all 60 treatment cycles in all 50 patients.

The patient showed a partial metabolic response (PMR) by Lugano criteria documented by PET CT scan within 14 days of treatment with blinatumomab. This rapid kinetic of response had never been observed before at that therapeutic dose in Non Hodgkin Lymphoma (NHL). Meanwhile LDH has gone back to baseline.

1. A method of treating Burkitt lymphoma or leukemia in a patient, the method comprising administering to said patient a composition comprising a CD19×CD3 bispecific antibody in at least one treatment cycle, wherein the treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time.

2. The method of claim 1, wherein the second period of time exceeds 18 days.

3. The method of claim 1 or 2, wherein the second period of time is between 18 and 60 days, with 24 days being preferred.

4. The method of any one of claims 1 to 3, further comprising administering after the first treatment cycle at least a second treatment cycle.

5. The method of claim 4, wherein the second treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time.

6. The method of claim 5, wherein the second period of time exceeds 18 days.

7. The method of claim 5 or 6, wherein the second period of time is between 18 and 60 days, with 24 days being preferred.

8. The method of any one of claims 4 to 7, wherein the second treatment cycle is initiated at least 5 days after completing the first treatment cycle, preferably 10 days after completing the first treatment cycle.

9. The method of any one of claims 4 to 8, further comprising administering after the first and second treatment cycle at least a third treatment cycle, preferably five additional treatment cycles.

10. The method of any one of the preceding claims, wherein the route of administration of the first and the second dose of the CD19×CD3 bispecific antibody is intravenous, preferably continuous intravenous.

11. The method of any one of the preceding claims, wherein the Burkitt lymphoma or leukemia is relapsed and/or refractory Burkitt lymphoma or leukemia.

12. The method of any one of the preceding claims, wherein the Burkitt lymphoma or leukemia is a CD19 positive Burkitt lymphoma or leukemia.

13. The method of any one of the preceding claims, wherein the CD19×CD3 bispecific antibody is a bispecific single chain antibody.

14. The method of claim 13, wherein said antibody is MT103/AMG103.

15. The method of any one of the preceding claims, further comprising administering pentosane polysulfate (PPS) prior to the first dose and prior to the second dose of the CD19×CD3 bispecific antibody of each treatment cycle to said patient.

16. The method of claim 15, wherein PPS is administered between 24 and 2 hours prior to the first dose and prior to the second dose of the CD19×CD3 bispecific antibody, preferably 24 hours prior to the first dose of the CD19×CD3 bispecific antibody.

17. The method of claim 15 or 16, wherein PPS administration is continued for at least 3 consecutive days after administration of the first dose and the second dose of the CD19×CD3 bispecific antibody.

18. The method of claim 17, wherein PPS administration is continued for 7, 8, 9 or 10 consecutive days after administration of the first dose of the CD19×CD3 bispecific antibody, preferably for 7 consecutive days after administration of the first dose of the CD19×CD3 bispecific antibody.

19. The method of any one of claims 15 to 18, wherein PPS is administered in a dose between 100 and 600 mg per day.

20. The method of claim 19, wherein PPS is administered in a dose of 300 mg per day.

21. The method of claim 20, wherein PPS is administered in three doses of 100 mg per day.

22. The method of any one of claims 15 to 21, wherein PPS is administered orally to said patient.

23. The method of any one of the preceding claims, wherein the treatment results in a MRD negative status of the Burkitt lymphoma or leukemia patient.

24. The method of any one of the preceding claims, wherein the patient is characterized by a B:T cell ratio of less than 1:8.

25. The method of any one of the preceding claims, wherein said patient is a mammal, preferably a primate, most preferably a human being.

26. A composition comprising a CD19×CD3 bispecific antibody for use in a method of treating Burkitt lymphoma or leukemia in a patient, wherein the method comprises administering to said patient the composition in at least one treatment cycle, wherein the treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time.

27. The composition for use of claim 26, wherein the second period of time exceeds 18 days.

28. The composition for use of claim 26 or 27, wherein the second period of time is between 18 and 60 days, with 24 days being preferred.

29. The composition for use of any one of claims 26 to 28, further comprising administering after the first treatment cycle at least a second treatment cycle.

30. The composition for use of claim 29, wherein the second treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time.

31. The composition for use of claim 30, wherein the second period of time exceeds 18 days.

32. The composition for use of claim 30 or 31, wherein the second period of time is between 18 and 60 days, with 24 days being preferred.

33. The composition for use of any one of claims 29 to 32, wherein the second treatment cycle is initiated at least 5 days after completing the first treatment cycle, preferably 10 days after completing the first treatment cycle.

34. The composition for use of any one of claims 29 to 33, further comprising administering after the first and second treatment cycle at least a third treatment cycle, preferably five additional treatment cycles.

35. The composition for use of any one of the preceding claims, wherein the route of administration of the first and the second dose of the CD19×CD3 bispecific antibody is intravenous, preferably continuous intravenous.

36. The composition for use of any one of the preceding claims, wherein the Burkitt lymphoma or leukemia is relapsed and/or refractory Burkitt lymphoma or leukemia.

37. The composition for use of any one of the preceding claims, wherein the Burkitt lymphoma or leukemia is a CD19 positive Burkitt lymphoma or leukemia.

38. The composition for use of any one of the preceding claims, wherein the CD19×CD3 bispecific antibody is a bispecific single chain antibody.

39. The composition for use of claim 38, wherein said antibody is MT103/AMG103.

40. The composition for use of any one of the preceding claims, further comprising administering pentosane polysulfate (PPS) prior to the first dose and prior to the second dose of the CD19×CD3 bispecific antibody of each treatment cycle to said patient.

41. The composition for use of claim 40, wherein PPS is administered between 24 and 2 hours prior to the first dose and prior to the second dose of the CD19×CD3 bispecific antibody, preferably 24 hours prior to the first dose and prior to the second dose of the CD19×CD3 bispecific antibody.

42. The composition for use of claim 40 or 41, wherein PPS administration is continued for at least 3 consecutive days after administration of the first dose and the second dose of the CD19×CD3 bispecific antibody.

43. The composition for use of claim 42, wherein PPS administration is continued for 7, 8, 9 or 10 consecutive days after administration of the first dose of the CD19×CD3 bispecific antibody, preferably for 7 consecutive days after administration of the first dose of the CD19×CD3 bispecific antibody.

44. The composition for use of any one of claims 40 to 43, wherein PPS is administered in a dose between 100 and 600 mg per day.

45. The composition for use of claim 44, wherein PPS is administered in a dose of 300 mg per day.

46. The composition for use of claim 45, wherein PPS is administered in three doses of 100 mg per day.

47. The composition for use of any one of claims 40 to 46, wherein PPS is administered orally to said patient.

48. The composition for use of any one of the preceding claims, wherein the treatment results in a MRD negative status of the Burkitt lymphoma or leukemia patient.

49. The composition for use of any one of the preceding claims, wherein the patient is characterized by a B:T cell ratio of less than 1:8.

50. The composition for use of any one of the preceding claims, wherein said patient is a mammal, preferably a primate, most preferably a human being.

51. Use of a composition comprising a CD19×CD3 bispecific antibody for the manufacture of a medicament for the treatment of Burkitt lymphoma or leukemia in a patient, the treatment comprising administering to said patient the composition in at least one treatment cycle, wherein the treatment cycle comprises administering a first dose of 15 μg/m²/d of the CD19×CD3 bispecific antibody for a first period of time of 4 days, and administering after the first dose a second dose of 60 μg/m²/d of the CD19×CD3 bispecific antibody for a second period of time, wherein the second period of time exceeds the first period of time.


SEQUENCE LISTING (23 sequences)

SEQ ID NO: 1 - protein, 498 aa, Artificial Sequence
  CD19xCD3 bispecific single chain antibody
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 2 - DNA, 1494 bp, Artificial Sequence
  CD19xCD3 bispecific single chain antibody
gatatccagctgacccagtctccagcttctttggctgtgtctctagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggagggaccaagctcgagatcaaaggtggtggtggttctggcggcggcggctccggtggtggtggttctcaggtgcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggccaagggaccacggtcaccgtctcctccggaggtggtggatccgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagtcgaaggtggaagtggaggttctggtggaagtggaggttcaggtggagtcgacgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaa

SEQ ID NO: 3 - protein, 124 aa, Artificial Sequence
  VH anti CD19
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 4 - DNA, 372 bp, Artificial Sequence
  VH anti CD19
caggtgcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggccaagggaccacggtcaccgtctcctcc

SEQ ID NO: 5 - protein, 111 aa, Artificial Sequence
  VL anti CD19
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 6 - DNA, 333 bp, Artificial Sequence
  VL anti CD19
gatatccagctgacccagtctccagcttctttggctgtgtctctagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggagggaccaagctcgagatcaaa

SEQ ID NO: 7 - protein, 119 aa, Artificial Sequence
  VH anti CD3
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 8 - DNA, 357 bp, Artificial Sequence
  VH anti CD3
gatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctca

SEQ ID NO: 9 - protein, 106 aa, Artificial Sequence
  VL anti CD3
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 10 - DNA, 318 bp, Artificial Sequence
  VL anti CD3
gacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaa

SEQ ID NO: 11 - protein, 10 aa, artificial
  CD3 CDR-H1
GYTFTRYTMH

SEQ ID NO: 12 - protein, 17 aa, artificial
  CD3 CDR-H2
YINPSRGYTNYNQKFKD

SEQ ID NO: 13 - protein, 10 aa, artificial
  CD3 CDR-H3
YYDDHYCLDY

SEQ ID NO: 14 - protein, 10 aa, artificial
  CD3 CDR-L1
RASSSVSYMN

SEQ ID NO: 15 - protein, 7 aa, artificial
  CD3 CDR-L2
DTSKVAS

SEQ ID NO: 16 - protein, 9 aa, artificial
  CD3 CDR-L3
QQWSSNPLT

SEQ ID NO: 17 - protein, 10 aa, artificial
  CD19 CDR-H1
GYAFSSYWMN

SEQ ID NO: 18 - protein, 17 aa, artificial
  CD19 CDR-H2
QIWPGDGDTNYNGKFKG

SEQ ID NO: 19 - protein, 15 aa, artificial
  CD19 CDR-H3
RETTTVGRYYYAMDY

SEQ ID NO: 20 - protein, 15 aa, artificial
  CD19 CDR-L1
KASQSVDYDGDSYLN

SEQ ID NO: 21 - protein, 7 aa, artificial
  CD19 CDR-L2
DASNLVS

SEQ ID NO: 22 - protein, 9 aa, artificial
  CD19 CDR-L3
QQSTEDPWT

SEQ ID NO: 23 - protein, 5 aa, Artificial Sequence
  Peptide Spacer
GGGGS